(19) United States
(12) Patent Application Publication
(10) Pub. No.: US 2026/0216375 A1
(43) Pub. Date: Jul. 30, 2026

(54) PROCESS FOR PRODUCING DNA FORMULATIONS WITH LIPOPOLYMER DELIVERY SYSTEMS

(71) Applicant: IMUNON, INC., Lawrenceville, NJ (US)

(72) Inventors: Majed Mohd MATAR, Madison, AL (US); Brian Jeffrey SPARKS, Huntsville, AL (US); Alanna SMITH, Madison, AL (US); Jennifer RICE, Grant, AL (US); Khursheed ANWER, Madison, AL (US)

(21) Appl. No.: 19/152,274

(22) PCT Filed: Jan. 30, 2024
(86) PCT No.: PCT/US2024/013589

§ 371 Date: Jul. 30, 2025

Related U.S. Application Data
(60) Provisional application No. 63/482,441, filed on Jan. 31, 2023.

(51) Int. Cl. A61K 48/00; A61K 9/51; A61K 38/20; A61P 35/00

(52) U.S. Cl. CPC A61K 48/005; A61K 9/5123; A61K 9/5146; A61K 38/208; A61P 35/00

(57) ABSTRACT

Certain aspects of the invention are directed to methods of making a concentrated nucleic acid composition comprising: (a) combining (i) a DNA plasmid comprising a nucleic acid encoding a human IL-12 polypeptide, (ii) a cationic lipopolymer comprising polyethylene glycol (PEG)-polyethylenimine (PEI)-cholesterol (PPC), and (iii) a filler excipient in an aqueous medium, wherein the nucleic acid complexes with the cationic lipopolymer, thereby forming a nucleic acid mixture having a concentration of at least 0.15 mg/mL; and (b) concentrating the nucleic acid mixture of (a) by tangential flow filtration to form a concentrated nucleic acid composition having a concentration of at least 0.75 mg/mL, wherein the concentrated nucleic acid composition is suitable for pharmaceutical use, storage at −20° C. or less, 4° C. or less, and/or lyophilization. The invention further relates to pharmaceutical compositions prepared using the disclosed methods and methods of using the same.

### CROSS-REFERENCE TO RELATED APPLICATIONS

This application claims the benefit of U.S. Provisional Application No. 63/482,441, filed Jan. 31, 2023, which is hereby incorporated by reference in its entirety.

### FIELD OF THE DISCLOSURE

The disclosure involves the fields of molecular biology and biochemistry. For example, the disclosure relates to methods and processes for preparing formulations comprising a nucleic acid and a lipopolymer.

### BACKGROUND

GEN-1 is a gene-based cancer therapy comprising a human IL-12 gene expression plasmid and a synthetic lipopolymer delivery system (Thaker, Premal H. et al., Future Oncol. (2019) 15 (4), 421-438). GEN-1 can be delivered intraperitoneally (i.p.) to produce local and persistent levels of IL-12 at the tumor site in patients with advanced ovarian cancer. GEN-1 can be administered alone or in combination with chemotherapy. GEN-1 includes a plasmid vector encoding the p35 and p40 subunits of human IL-12 genes, each under the control of a cytomegalovirus (CMV) promotor, and a synthetic lipopolymer delivery system, polyethylene glycol (PEG)-polyethylenimine (PEI)-cholesterol (PPC). Currently, GEN-1 is formulated as a lyophilized powder that can be reconstituted at the bedside to doses up to about 0.5 mg/mL.

Pharmaceuticals containing DNA that self-assembles into nanoparticles often exhibit poor stability, particularly when the formulation is an aqueous suspension. In such formulations, DNA with synthetic vectors will typically aggregate over time, especially at concentrations required for optimal dosing in a clinical setting. Such formulations are often difficult to prepare at DNA concentrations greater than 0.3 mg/mL, which limits their commercial applications, especially for local delivery where volume constraints limit flexible dosing. DNA aggregation reduces or eliminates the activity of the DNA and therefore can make the composition unsuitable for use in treatment.

Freeze-drying is a useful method for improving long-term stability of a number of drug pharmaceuticals. However, this process presents challenges for drying DNA complexes with synthetic vectors as it tends to alter their physicochemical properties and results in aggregation and loss of transfection upon reconstitution.

Approaches have been attempted to prevent formulation aggregation and damage during lyophilization. In some cases, lyophilization of DNA complexes in the presence of a cryoprotectant such as low molecular weight sugars, dextrans, and polyethylene glycol may provide better stability to the product, but that approach does not appear to improve dosing flexibility. Addition of sugars is often the most commonly used approach for this purpose. Many of the test sugars have been found to prevent formulation damage and particle aggregation to some extent, but the quality of this effect varies with the type of sugar and the delivery vector used.

Although lyophilization provides some improvement in formulation shelf life, the conditions required to produce lyophilized DNA products allow for only limited pharmaceutical applications. Even with the most effective lyoprotectant sugars, a very high sugar/DNA molar ratio (typically greater than 1000:1) is required for stability. As a result, the lyophilized product often must be diluted by a very large factor to obtain an isotonic formulation, which results in a drop in the final DNA concentration to the pre-lyophilized DNA concentration. For many cationic carriers the final DNA concentration may typically be about 0.1-0.2 mg/mL, and often below 0.1 mg/mL.

A method to achieve compositions with higher DNA concentrations (e.g., 0.15 mg/mL to 0.5 mg/mL) was disclosed in U.S. Pat. No. 9,144,546. This process can yield 6 mg of lyophilized product/vial reconstituted to 0.5 mg/mL in 12 mL sterile water for injection (WFI). For example, a clinical dose of about 100 mg/m² requires about 180 mg DNA in an average 1.8 m² surface area subject. A 180 mg DNA dose takes 30 vials (50 mL vials) to reconstitute each vial carrying 6 mg DNA to reconstitute. This is a commercially challenging preparation.

Although low concentration formulations are sufficient for in vitro studies, their clinical application may be limited due to high volume requirement for optimal dosing.

There is a need for improved methods for preparation of DNA therapies, e.g., for concentrated, temperature stable, and biologically active DNA formulations.

### SUMMARY

Certain aspects of the disclosure are directed to a method of making a concentrated nucleic acid composition comprising: (a) combining (i) a DNA plasmid comprising a nucleic acid encoding a human IL-12 polypeptide, (ii) a cationic lipopolymer comprising polyethylene glycol (PEG)-polyethylenimine (PEI)-cholesterol (PPC), and (iii) a filler excipient in an aqueous medium, wherein the nucleic acid complexes with the cationic lipopolymer, thereby forming a nucleic acid mixture having a concentration of at least 0.10 to 0.2 mg/mL (e.g., at least 0.15 mg/mL, e.g., 0.15 to 0.2 mg/mL); and (b) concentrating the nucleic acid mixture by tangential flow filtration to form a concentrated nucleic acid composition having a concentration of at least 0.7 mg/mL to 0.8 mg/mL (e.g., at least 0.75 mg/mL, e.g., 0.75 to 1 mg/mL), wherein the concentrated nucleic acid composition is suitable for pharmaceutical use, storage at about 4° C. or less, storage at about −20° C. or less, and/or lyophilization.

In some aspects, the percent recovery of nucleic acid complexed with cationic lipopolymer after tangential flow filtration is at least 75%, or at least 80%, or at least 85%, or at least 90%, or at least 95%, or 100%.

In some aspects, the method further comprises storing the concentrated nucleic acid composition at about 4° C. or less for at least 24 hours, at least one week, at least one month, at least one year, or at least 30 months.

In some aspects, the method further comprises storing the concentrated nucleic acid composition at about −20° C. or less for at least 24 hours, at least one week, at least one month, or at least one year.

In some aspects, the concentrated nucleic acid is storage stable. In some aspects, one or more of the following is stable at 4° C. or less for at least 30 months: particle size, DNA concentration, PPC concentration, PPC/DNA ratio, osmolality, and/or pH.

In some aspects, the nucleic acid mixture has a concentration of at least 0.15 mg/mL. In some aspects, the nucleic acid mixture has a concentration of about 0.15 mg/mL to 0.2 mg/mL.

In some aspects, the concentration nucleic acid composition has a concentration of at least 0.75 mg/mL. In some aspects, the concentration nucleic acid composition has a concentration of about 0.75 mg/mL to 1 mg/mL.

In some aspects, the method further comprises (c) lyophilizing the concentrated nucleic acid composition having a concentration of at least 0.7 mg/mL to 0.8 mg/mL (e.g., at least 0.75 mg/mL) in a volume of 20-40 mL, thereby forming a lyophilized formulation comprising at least 10 mg, at least 15 mg, at least 20 mg, at least 25 mg, or at least 30 mg of the DNA plasmid (e.g., about 10 to 50 mg).

In some aspects, the lyophilized formulation comprises 10-50 mg (e.g., about 30 mg) of the DNA plasmid.

In some aspects, the method further comprises (d) reconstituting the lyophilized formulation in a diluent (e.g., water, 5% dextrose, or saline) thereby forming a reconstituted composition.

In some aspects, the nucleic acid mixture comprises GEN-1 nanoparticles.

In some aspects, the ratio of amine nitrogen in the cationic polymer backbone to phosphate in the nucleic is from about 10:1 to about 100:1.

In some aspects, the filler excipient comprises a sugar, a sugar alcohol, a starch, a cellulose, or combinations thereof. In some aspects, the filler excipient comprises one or more of lactose, sucrose, trehalose, dextrose, galactose, mannitol, maltitol, maltose, sorbitol, xylitol, mannose, glucose, fructose, polyvinyl pyrrolidone, glycine, maltodextrin, hydroxymethyl starch, gelatin, sorbitol, ficol, sodium chloride, calcium phosphate, calcium carbonate, and/or polyethylene glycol. In some aspects, the filler excipient comprises lactose.

Certain aspects of the disclosure provides methods whereby low concentration nucleic acid compositions (e.g., 0.15 mg/mL) can be processed using a method comprising a tangential flow filtration (TFF) step to prepare a concentrated (e.g., 0.30 mg/mL to 0.75 mg/mL or higher) nucleic acid composition without affecting the physico-chemical or biological properties of the nucleic acid or nucleic acid composition. In some aspects, nucleic acid composition comprises a DNA plasmid comprising a polynucleotide encoding a therapeutic protein (e.g., a hIL-12 coding nucleic acid) and a lipopolymer (e.g., PPC). In some aspects, the nucleic acid composition comprises GEN-1. In some aspects, GEN-1 can be concentrated according to the methods of the disclosure into a solution comprising 0.30 mg/mL to 0.75 mg/mL.

In some aspects, the concentrated nucleic acid composition (pre-lyophilized) at a concentration (e.g., 0.30 mg/mL to 0.75 mg/mL or higher) can be used directly as a dosing regimen for administration to a subject. In some aspects, the concentrated nucleic acid composition can be stored at −20° C.

In some aspects, nucleic acid composition can be lyophilized according to the disclosure to provide a concentrated lyophilized nucleic acid composition comprising 10 mg to 100 mg (e.g., 10 mg to 100 mg, 10 mg to 90 mg, 10 mg to 80 mg, 10 mg to 70 mg, 10 mg to 60 mg, 10 mg to 50 mg, 10 mg to 40 mg, or 10 mg to 30 mg) of DNA. In some aspects, nucleic acid composition can be lyophilized according to the disclosure to provide a concentrated lyophilized nucleic acid composition comprising 10 mg to 50 mg (e.g., 10 mg to 40 mg, 15 mg to 30 mg, 20 mg to 40 mg, 25 mg to 40 mg, 25 mg to 35 mg, or about 30 mg) of DNA. In some aspects, the concentrated lyophilized nucleic acid composition can be stably stored (e.g., for at 1 year, at least two years, at least three years).

In some aspects, the concentrated lyophilized nucleic acid composition can be reconstituted (e.g., in water, 5% dextrose, or saline). In some aspects, the concentrated nucleic acid compositions allow for a wide range of dosing regimens in vivo after reconstitution of lyophilized composition.

In some aspects, the concentrated lyophilized nucleic acid composition can be reconstituted at 0.5 mg/mL, or 1 mg/mL, or 1.5 mg/mL, 2 mg/mL, or 2.5 mg/mL, or 3 mg/mL, or 3.5 mg/mL, or 4 mg/mL, or 4.5 mg/mL, or 5 mg/mL, or 5.5 mg/mL, or 6 mg/mL, or 6.5 mg/mL, or 7 mg/mL, or 7.5 mg/mL, or 8 mg/mL, or 8.5 mg/mL, or 9 mg/mL, or 9.5 mg/mL, or 10 mg/mL.

Certain aspects of the disclosure are directed to the concentrated nucleic acid compositions (pre-lyophilization and post-lyophilization) prepared according to the methods disclosed herein.

Certain aspects of the disclosure are directed to a pharmaceutical composition comprising a concentrated nucleic acid compositions (pre-lyophilization and post-lyophilization) prepared according to the methods disclosed herein. In some aspects, the pharmaceutical composition comprises the concentrated nucleic acid composition or reconstituted composition prepared according the methods comprising TFF disclosed herein.

In some aspects, the pharmaceutical composition comprises greater than 6 mg (e.g., 10-100 mg, 10-90 mg, 10-80 mg, 10-70 mg, 10-60 mg, 10-50 mg, 10-40 mg, 20-50 mg, 20-45 mg, 20-40 mg, 20-25 mg, or 25-35 mg) of DNA plasmid (complexed with PPC) in a volume of less than 70 mL (e.g., 20-70 mL, 20-60 mL, 20-50 mL, or 20-45 mL) of diluent. In some aspects, the pharmaceutical composition comprises greater than 6 mg (e.g., 10-50 mg, 10-40 mg, 20-50 mg, 20-25 mg, 20-40 mg, or 25-35 mg) of DNA plasmid (complexed with PPC) in a volume of less than 50 mL (e.g., 20-45 mL) of diluent. In some aspects, the pharmaceutical composition comprises about 30 mg of DNA plasmid (complexed with PPC) in a volume of 20-45 mL (e.g., about 40 mL) of diluent (e.g., water, 5% dextrose, or saline). In some aspects, the pharmaceutical composition comprises about 50 mg of DNA plasmid (complexed with PPC) in a volume of 20-45 mL (e.g., about 20 mL) of diluent (e.g., water, 5% dextrose, or saline).

In some aspects, the pharmaceutical composition is in a container which can hold up to 100 mL, 150 mL, 200 mL, 250 mL or 500 mL (e.g., a 250 mL vial) comprising 10-50 mg, 10-40 mg, 20-50 mg, 20-45 mg, 20-40 mg, 20-25 mg, or 25-35 mg of DNA in solution (e.g., 200 mL), wherein the concentration of DNA is at least 0.7 mg/mL to 0.8 mg/mL (e.g., at least 0.75 mg/mL).

In some aspects, the pharmaceutical composition is used to prepare a dosage of 100-200 mg (e.g., 180 mg) of plasmid DNA (complexed with PPC) in less than 300 mL (e.g., 200-300 mL) of diluent (e.g., water, 5% dextrose, or saline).

Certain aspects of the disclosure are directed to a kit comprising a first vial (e.g., a 250 mL vial) comprising a nucleic acid mixture comprising (i) a DNA plasmid comprising a nucleic acid encoding a human IL-12 polypeptide, (ii) a cationic lipopolymer comprising polyethylene glycol (PEG)-polyethylenimine (PEI)-cholesterol (PPC), and (iii) a filler excipient in an aqueous medium, wherein the nucleic acid is complexed with the cationic lipopolymer and the nucleic acid mixture comprises at least 25 mg to 50 mg (e.g., about 30 mg) of the DNA plasmid, and optionally further comprising a second vial comprising a diluent (e.g., about 20-45 mL).

Certain aspects of the disclosure are directed to a kit comprising a vial comprising a nucleic acid mixture comprising (i) a DNA plasmid comprising a nucleic acid encoding a human IL-12 polypeptide, (ii) a cationic lipopolymer comprising polyethylene glycol (PEG)-polyethylenimine (PEI)-cholesterol (PPC), and (iii) a filler excipient in an aqueous medium, wherein the nucleic acid is complexed with the cationic lipopolymer; and wherein the nucleic acid mixture is in an aqueous solution at a concentration of at least 0.7 mg/mL to 0.8 mg/L (e.g., at least 0.75 mg/mL).

In some aspects, the vial (e.g., first vial and/or second vial) can hold up to 100 mL, 150 mL, 200 mL, 250 mL or 500 mL (e.g., a 250 mL vial). In some aspects, the vial comprises a volume of 200-300 mL (e.g., about 200 mL) of diluent (e.g., water, 5% dextrose, or saline). In some aspects, the vial comprises 10-50 mg, 10-40 mg, 20-50 mg, 20-25 mg, 20-40 mg, or 25-35 mg (e.g., about 30 mg) of the DNA plasmid (complexed with PPC). In some aspects, the first vial and/or second vial can hold up to about 100 mL, about 150 mL, about 200 mL, about 250 mL or about 500 mL (e.g., a 250 mL vial). In some aspects, the second vial comprises a volume of about 200-300 mL (e.g., about 200 mL) of diluent (e.g., water, 5% dextrose, or saline). In some aspects, the first vial comprises about 10-50 mg, about 10-40 mg, about 20-50 mg, about 20-25 mg, about 20-45 mg, about 20-40 mg, or about 25-35 mg (e.g., about 30 mg) of the DNA plasmid (complexed with PPC).

In some aspects, the kit can be stored at −20° C. or less.

Certain aspects of the disclosure are directed to a method, comprising a mixture of a cationic lipopolymer and at least about 10 mg/mL of a nucleic acid, where the mixture is suspended in an isotonic solution. The cationic lipopolymer comprises a cationic polymer backbone having cholesterol and polyethylene glycol groups independently covalently attached thereto. The molar ratio of cholesterol to cationic polymer backbone is within a range of from about 0.1 to about 10, and the molar ratio of polyethylene glycol to cationic polymer backbone is within a range of from about 0.1 to about 10.

The method further may include a filler excipient. In certain aspects, the mixture of nucleic acid and lipopolymer forms a complex.

In certain aspects, the method comprises condensing of the nucleic acid through tangential flow filtration.

In another aspect, the disclosure provides a method for lyophilization of a nucleic acid and a lipopolymer. The lyophilization method of the disclosure comprises a mixture of a filler excipient, a nucleic acid condensed, and a cationic lipopolymer. As noted above, the cationic lipopolymer includes a cationic polymer backbone having cholesterol and polyethylene glycol covalently attached thereto, and wherein the molar ratio of cholesterol to cationic polymer backbone is within a range of from about 0.1 to about 10, and the molar ratio of polyethylene glycol to cationic polymer backbone is within a range of from about 0.1 to about 10. In certain aspects, the lyophilization method produces at least about 30 mg of nucleic acid and lipopolymer.

In another aspect, the disclosure provides a method, comprising a mixture of a cationic lipopolymer and at least about 0.75 mg/mL of a nucleic acid, where the mixture is suspended in an isotonic solution. The cationic lipopolymer comprises a cationic polymer backbone having cholesterol and polyethylene glycol groups independently covalently attached thereto. The molar ratio of cholesterol to cationic polymer backbone is within a range of from about 0.1 to about 10, and the molar ratio of polyethylene glycol to cationic polymer backbone is within a range of from about 0.1 to about 10. The method further may include a filler excipient. In certain aspects, the mixture of nucleic acid and lipopolymer forms a complex. In certain aspects, the method comprises condensing of the nucleic acid through tangential flow filtration. In certain aspects, the condensed nucleic acid is stored at a temperature of about 4° C. or less or at least about −20° C. (e.g., between about 4° C. to about −20° C.).

The disclosure additionally provides methods for using the compositions described herein in the treatment of diseases and/or disorders by, e.g., transfecting various cells and tissues. Certain aspects of the disclosure are directed to a method of treating cancer in a subject comprising administering a pharmaceutical composition prepared by the methods disclosed herein to a subject in need thereof. In some aspects, the pharmaceutical composition is formulated for intratumoral, intraperitoneal, intravesicle, intravenous, intra-arterial, intratracheal, intrahepaticportal, intracranial, intramuscular, or intraarticular administration. In some aspects, the cancer is ovarian cancer.

### BRIEF DESCRIPTION OF THE DRAWINGS

FIGS. 1A-1B show schematics of processes for preparation of lyophilized formulations of plasmid IL-12 DNA and PEI-PEG-cholesterol polymer (PPC). FIG. 1A shows a process using a static mixer. FIG. 1B shows a process further including a tangential flow filtration (TFF) step, and optional pre-lyophilization storage, resulting in production of 5× higher DNA/vial compared to the process of FIG. 1A.

FIGS. 2A-2E show graphs of particle size and osmolality of the nucleic acid formulation concentrated at 1×, 2×, 3×, 4×, and 5×. FIG. 2A shows particle size and osmolality of 1× and 5× concentrations. FIG. 2B shows particle size and polydispersity of the nucleic acid formulation concentrated at 1×, 2×, 3×, 4×, and 5× from a 10 L formulation scale. FIG. 2C shows osmolality and pH of the nucleic acid formulation concentrated at 1×, 2×, 3×, 4×, and 5× from a 10 L formulation scale. FIG. 2D shows osmolality of the nucleic acid formulation concentrated at 1×, 2×, 3×, 4×, and 5× from a 20 L formulation scale. FIG. 2E shows diameter of the nucleic acid formulation concentrated at 1×, 2×, 3×, 4×, and 5× from a 20 L formulation scale.

FIGS. 3A-3D show PEI-PEG-cholesterol polymer (PPC) and DNA recovery after tangential flow filtration (TFF) process of nucleic acid formulation concentrated at 1×, 2×, 3×, 4×, and 5×. FIG. 3A shows concentration of PPC and DNA ratios of 1× and 5× concentrations. FIG. 3B shows PPC and DNA concentrations of the nucleic acid formulation concentrated at 1×, 2×, 3×, 4×, and 5× from a 10 L formulation scale. FIG. 3C shows PPC/DNA percent recovery and PPC/DNA ratios of the nucleic acid formulation concentrated at 1×, 2×, 3×, 4×, and 5× from a 10 L formulation scale. FIG. 3D shows PPC and DNA concentrations of the nucleic acid formulation concentrated at 1×, 2×, 3×, 4×, and 5× from a 20 L formulation scale.

FIGS. 4A-4C show gel electrophoresis of nucleic acid formulation concentrated at 1×, 2×, 3×, 4×, and 5×. FIG. 4A shows gel electrophoresis of nucleic acid formulation concentrated at 1× and 5×. FIG. 4B shows gel electrophoresis with Dextran Sulfate and without Dextran Sulfate of nucleic acid formulation concentrated at 1×, 2×, 3×, 4×, and 5× from a 10 L formulation scale. FIG. 4C shows gel electrophoresis with Dextran Sulfate and without Dextran Sulfate of nucleic acid formulation concentrated at 1×, 2×, 3×, 4×, and 5× from a 20 L formulation scale.

FIGS. 5A-5D show in vitro expression levels of IL-12 in COS-1 cells from the nucleic acid formulation concentrated at 1×, 2×, 3×, 4×, and 5×. FIG. 5A shows expression levels of IL-12 at different doses from 1× and 5× concentrations. FIG. 5B show expression levels of IL-12 at different doses from 1×, 2×, 3×, 4×, and 5× concentrations from a 8.5 L formulation scale. FIG. 5C show expression levels of IL-12 at different doses from 1×, 2×, 3×, 4×, and 5× concentrations from a 10 L formulation scale. FIG. 5D show expression levels of IL-12 at different doses from 1×, 2×, 3×, 4×, and 5× concentrations from a 20 L formulation scale.

FIGS. 6A-6B show graphs of hIL-12 expression levels (total pg (FIG. 6A) and pg/mL (FIG. 6B)) in tumor bearing mice following treatments with nucleic acid formulation concentrated at 1×, 2×, 3×, 4×, and 5×.

FIGS. 7A-7B show graphs of hIL-12 expression levels (total pg (FIG. 7A) and pg/mL (FIG. 7B)) in tumor bearing mice following nucleic acid formulation treatment from different lots.

FIGS. 8A-8I show graphs of nucleic acid/cationic lipopolymer composition after up to 30 months of storage at 4° C. and −20° C. FIGS. 8A-8F shows particle size, DNA concentration, PPC concentration, PPC/DNA ratio, osmolality, and pH, respectively, of a reference standard stored at −80° C. and 5× concentrations stored for up to 30 months at 4° C. and −20° C. FIG. 8G shows expression levels of IL-12 at different doses from a reference standard stored at −80° C., 1× concentration stored for up to 30 months at −20° C., and 5× concentrations stored for up to 30 months at 4° C. and −20° C. FIG. 8H shows relative potency of 5× concentrations stored for up to 30 months at 4° C. and −20° C., when compared to a reference standard stored at −80° C. FIG. 8I shows expression levels of IL-12 at different doses from 1× and 5× concentrations stored at the 30 month period at 4° C. and −20° C.

### DETAILED DESCRIPTION

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

As used herein, the term “nucleic acid” refers to DNA and RNA, as well as synthetic congeners thereof. In addition, the nucleic acid can be variable in size, ranging from oligonucleotides to chromosomes. Nucleic acids can be of human, animal, vegetable, bacterial, viral, or synthetic origin. They can be obtained by any technique known to a person skilled in the art.

“Coding sequence” or a sequence “encoding” a particular molecule (e.g., a therapeutic molecule) is a nucleic acid that is transcribed (in the case of DNA) or translated (in the case of mRNA) into polypeptide, in vitro or in vivo, when operably linked to an appropriate regulatory sequence, such as a promoter. The boundaries of the coding sequence are determined by a start codon at the 5′ (amino) terminus and a translation stop codon at the 3′ (carboxy) terminus. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. A transcription termination sequence will usually be located 3′ to the coding sequence.

As used herein, the term “promoter/regulatory sequence” refers to a nucleic acid sequence required to express a gene product operably linked to a promoter/regulatory sequence. The term “constitutive” promoter refers to a nucleotide sequence that, when operably linked to a polynucleotide encoding or specifying a gene product, results in the production of a gene product in the cell under most or all physiological conditions of the cell. The term “inducible” promoter means that when operably linked to a polynucleotide encoding a specified gene product, it basically results in the production of a gene in the cell only when the inducer corresponding to the promoter is present in the cell the nucleotide sequence of the product.

As used herein, the term “expression” refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). Expression of a gene produces a “gene product.”

As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the term “expression vector” refers to a vector comprising a recombinant polynucleotide comprising an expression control sequence operably linked to the nucleotide sequence to be expressed. The expression vector contains sufficient cis-acting elements for expression; other elements for expression can be provided by the host cell or in an in vitro expression system. Expression vectors include expression vectors known in the art, including cosmids, plasmids (for example, naked or contained in liposomes), and viruses incorporating recombinant polynucleotides (for example, lentivirus, retrovirus, adenovirus, and adeno-associated virus). In some aspects, the expression vector is a DNA plasmid.

As used herein, the term “operably linked” or “transcription control” refers to a functional linkage between a regulatory sequence and a heterologous nucleic acid sequence, which results in the expression of the latter. For example, when the first nucleic acid sequence and the second nucleic acid sequence are arranged in a functional relationship, the first nucleic acid sequence and the second nucleic acid sequence are operably linked. For example, if a promoter affects the transcription or expression of a coding sequence, the promoter is operably linked to the coding sequence. The operably linked DNA sequences may be adjacent to each other, and for example, in the case where two protein coding regions need to be linked, the DNA sequences are in the same reading frame.

As used herein, the term “transfer vector” refers to a composition containing an isolated nucleic acid and a substance that can be used to deliver the isolated nucleic acid to the inside of a cell. Many vectors are known in the art, including but not limited to linear polynucleotides, polynucleotides associated with ionic or amphiphilic compounds, plasmids, and viruses. The term transfer vector should also be interpreted to further include non-plasmid and non-viral compounds that facilitate the transfer of nucleic acids into cells, such as polylysine compounds, liposomes, and the like.

As used herein, the term “host cell” can be any type of cell, e.g., a primary cell, a cell in culture, or a cell from a cell line. In specific aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

As used herein, the terms “condensed nucleic acid” and “partially condensed nucleic acid” are used to refer to a nucleic acid that has been contacted with a cationic lipopolymer of the disclosure. In certain aspects, the condensed nucleic acid remains in contact with the cationic lipopolymer. Condensed nucleic acids typically occupy a significantly smaller volume than non-condensed nucleic acids. It is recognized, however, that the amount of condensed nucleic acid may vary with local environment (e.g., lipid as opposed to aqueous environment). In various aspects of the disclosure, the condensed nucleic acids are those in nanoparticles of nucleic acid or cationic lipopolymer having a size of from about 50 nm to about 300 nm, more preferably from about 50-200, and even more preferably from about 50-150 nm. “Partially condensed nucleic acid” refers to a nucleic acid that has been contacted with a cationic lipopolymer of the disclosure wherein the nucleic acid is less than fully condensed, yet still occupy a significantly smaller volume than non-condensed nucleic acid.

As used herein, the term “complex” means nucleic acid that is associated with lipopolymer, e.g., cationic lipopolymer. A complex that includes condensed nucleic acid and cationic lipopolymer will typically exist as particles, e.g., as a nanoparticle.

As used herein, the term “concentrated” refers to a composition whose dilution has been reduced. In some aspects, a concentrated composition comprises condensed DNA, e.g., in an isotonic solution.

As used herein, the term “polymeric backbone” refers to a collection of polymeric backbone molecules having a weight average molecular weight within the designated range. As such, when a molecule such as cholesterol is described as being covalently attached thereto within a range of molar ratios, it should be understood that such a ratio represents an average number of cholesterol molecules attached to the collection of polymeric backbone molecules. For example, if cholesterol is described as being covalently attached to a polymeric backbone at a molar ratio of 0.5, then, on average, one half of the polymeric backbone molecules will have cholesterol attached. As another example, if cholesterol is described as being covalently attached to a polymeric backbone at a molar ratio of 1.0, then, on average, one cholesterol molecule will be attached to each of the polymeric backbone molecules. In reality, however, it should be understood that in this case some polymeric backbone molecules may have no cholesterol molecules attached, while other polymeric backbone molecules may have multiple cholesterol molecules attached, and that it is the average number of attached cholesterol molecules from which the ratio is derived. The same reasoning applies to the molar ratio of polyethylene glycol to the polymeric backbone.

As used herein, the term “peptide” can refer to a natural or synthetic molecule comprising two or more amino acids linked by the carboxyl group of one amino acid to the alpha amino group of another. A peptide of the disclosure is not limited by length, and thus “peptide” can include polypeptides and proteins.

As used herein, the terms “covalent” and “covalently” refer to chemical bonds whereby electrons are shared between pairs of atoms.

As used herein, “composition” refers to a mixture of two or more compounds, elements, or molecules. In some aspects, the term “composition” can be used to refer to a mixture of a nucleic acid and a delivery system (e.g., a cationic lipopolymer).

As used herein, “aqueous medium” or “aqueous solution” refers to a solution or mixture in which water is the carrier or solvent.

The term “pharmaceutically acceptable” as used herein refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “excipient” refers to any substance, not itself a therapeutic agent, which can be used in a composition for delivery of an active therapeutic agent to a subject or combined with an active therapeutic agent (e.g., to create a pharmaceutical composition) to improve its handling or storage properties or to permit or facilitate formation of a dose unit of the composition. Excipients include, but are not limited to, solvents, penetration enhancers, wetting agents, antioxidants, lubricants, emollients, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug. The excipient can be an inert substance, an inactive substance, and/or a not medicinally active substance. The excipient can serve various purposes.

A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21st edition, Gennaro, Ed., Lippincott Williams & Wilkins (2005).

As used herein, “N: P ratio” refers to the molar ratio of amine nitrogens in the functionalized cationic lipopolymer and the phosphate groups in the nucleic acid.

As used herein, “physicochemical properties” refers to various properties such as, without limitation, particle size and surface charge of nucleic acid complexes with a cationic polymer, pH and osmolarity of the particle solution, etc.

As used herein, the terms “transfecting” and “transfection” refer to the transportation of nucleic acids from the environment external to a cell to the internal cellular environment, e.g., the cytoplasm and/or cell nucleus. In some aspects, it is to be understood that nucleic acids can be delivered to cells either after being encapsulated within or adhering to one or more cationic polymer/nucleic acid complexes or being entrained therewith. Particular transfecting instances deliver a nucleic acid to a cell nucleus. Without being bound by any particular theory, it is to be understood that nucleic acids may be delivered to cells either after being encapsulated within or adhering to one or more cationic polymer/nucleic acid complexes or being entrained therewith. Particular transfecting instances deliver a nucleic acid to a cell nucleus. Nucleic acids include DNA and RNA as well as synthetic congeners thereof. Such nucleic acids include missense, antisense, nonsense, as well as protein producing nucleotides, on and off and rate regulatory nucleotides that control protein, peptide, and nucleic acid production. In particular, but not limited to, they can be genomic DNA, cDNA, mRNA, tRNA, rRNA, hybrid sequences or synthetic or semi-synthetic sequences, and of natural or artificial origin. In addition, the nucleic acid can be variable in size, ranging from oligonucleotides to chromosomes. These nucleic acids can be of human, animal, vegetable, bacterial, viral, or synthetic origin. They can be obtained by any technique known to a person skilled in the art.

As used herein, “vial” or “container” refers to vessel capable of containing or storing a substance. In some aspects, the vial can be shaped like a tube or bottle and have a flat or rounded bottom. In some aspects, the vial can be used to contain or store liquids. In some aspects, the vial can be made of glass or plastic. In some aspects, the vial can be sealed with a lid or not have a lid. In some aspects, the vial can hold a volume of at least 50 mL to 500 mL.

As used herein, “subject” refers to a mammal, particularly, a mammal that may benefit from the administration of a composition of this disclosure. Examples of subjects include humans, and can also include other mammals such as mice, horses, pigs, cattle, dogs, cats, rabbits, and aquatic mammals.

As used herein, the terms “administration,” “administering,” and “delivering” refer to the manner in which a composition is presented to a subject or cell. Administration to a subject can be accomplished by various art-known routes such as oral, parenteral, transdermal, inhalation, implantation, etc. In some aspects, an oral administration can be achieved by swallowing, chewing, sucking of an oral dosage form comprising the composition. In some aspects, parenteral administration can be achieved by injecting a composition intravenously, intra-arterially, intramuscularly, intraarticularly, intrathecally, intraperitoneally, subcutaneously, intratumorally, etc. Injectables for such use can be prepared in conventional forms, either as a liquid solution or suspension, or in a solid form that is suitable for preparation as a solution or suspension in a liquid prior to injection, or as an emulsion. Additionally, transdermal administration can be accomplished by applying, pasting, rolling, attaching, pouring, pressing, rubbing, etc., of a transdermal composition onto a skin surface. These and additional methods of administration are well-known in the art. In one aspect, administration may include delivering a composition to a subject such that the composition circulates systemically and binds to a target cell to be taken up by endocytosis.

The term “tumor” as used herein refers to any mass of tissue that results from excessive cell growth or proliferation, either benign (non-cancerous) or malignant (cancerous), including pre-cancerous lesions.

The term “primary tumor”, as used herein, refers to the original, or first, tumor formed in the subject's body.

The term “metastasis”, “metastatic”, “secondary tumor”, or “metastatic tumor”, as used herein, refer to cancer (e.g., a tumor) formed by cancer cells derived from a primary cancer (e.g., tumor) that spread to further locations or areas of the body.

As used herein, the term “cancer” refers to a broad group of various diseases characterized by the uncontrolled growth of abnormal cells (e.g., malignant cells) in the body. Unregulated cell division and growth results in the formation of malignant tumors that invade neighboring tissues through local spread and can also metastasize to distant parts of the body through the lymphatic system or bloodstream. In some aspects, the methods of the present disclosure can be used to reduce the size of a primary tumor or a metastatic tumor, or treat a primary tumor or a metastatic tumor. The conditions that can be treated or prevented by the method of the present disclosure include, for example, various neoplasms, including benign or malignant tumors, various hyperplasias, and the like. The method of the present disclosure can achieve the inhibition and/or reversal of the undesirable hyperproliferative cell growth involved in such conditions. In some aspects, the cancer can be ovarian cancer.

As used herein, “ovarian cancer” refers to cancers arising in, or involving, the ovaries, e.g. in the epithelium of the ovaries. As used herein, the term “cancer” or “tumor” refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastases. Cancers which migrate from their original location and seed vital organs can eventually lead to the death of the subject through the functional deterioration of the affected organs. Ovarian cancer is typically treated by cytoreductive surgery (also referred to herein as “debulking”) followed by administration of chemotherapy.

As used herein, the term “combination therapy” means a therapy that includes more than one treatment (e.g., active agent or procedure). In some aspects, compositions of the combination therapy are formulated together in a single composition or as separate compositions.

The term “chemotherapy” or “chemotherapeutic agent” as used herein refers to a wide variety of chemotherapeutic agents that may be used in accordance with the present disclosure. The term “chemotherapy” refers to the use of drugs to treat cancer. A chemotherapeutic agent can connote a compound or composition that is administered in the treatment of cancer.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data can be expressed or presented herein in a range format. It is to be understood that such a range format may be used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

### II. Methods for Preparation of Nucleic Acid Compositions

Certain aspects of the disclosure are directed to a method of making a concentrated nucleic acid composition comprising: (a) combining (i) a DNA plasmid comprising a nucleic acid encoding a human IL-12 polypeptide, (ii) a cationic lipopolymer comprising polyethylene glycol (PEG)-polyethylenimine (PEI)-cholesterol (PPC), and (iii) a filler excipient in an aqueous medium, wherein the nucleic acid complexes with the cationic lipopolymer, thereby forming a nucleic acid mixture having a concentration of at least 0.15 mg/mL; and (b) concentrating the nucleic acid mixture of (a) by tangential flow filtration to form a concentrated nucleic acid composition having a concentration of at least 0.75 mg/mL, wherein the percent recovery of nucleic acid complexed with cationic lipopolymer after tangential flow filtration is at least 75%, or at least 80%, or at least 85%, or at least 90%, or at least 95%, or 100%, wherein the concentrated nucleic acid composition is suitable for pharmaceutical use, storage at −20° C. or less, storage at 4° C. or less, and/or lyophilization.

Certain aspects of the disclosure are directed to a method of making a concentrated nucleic acid composition comprising: (a) combining (i) a DNA plasmid comprising a nucleic acid encoding a human IL-12 polypeptide, (ii) a cationic lipopolymer comprising polyethylene glycol (PEG)-polyethylenimine (PEI)-cholesterol (PPC), and (iii) a filler excipient in an aqueous medium, wherein the nucleic acid complexes with the cationic lipopolymer, thereby forming a nucleic acid mixture having a concentration of at least about 0.15 mg/mL; and (b) concentrating the nucleic acid mixture of (a) by tangential flow filtration to form a concentrated nucleic acid composition having a concentration of at least about 0.75 mg/mL, wherein the percent recovery of nucleic acid complexed with cationic lipopolymer after tangential flow filtration is at least about 75%, at least about 80%, at least about 85%, at least about 90%, at least about 95%, or about 100%, wherein the concentrated nucleic acid composition is suitable for pharmaceutical use, storage at about −20° C. or less, storage at about 4° C. or less, and/or lyophilization.

In some aspects, the method further comprises storing the concentrated nucleic acid composition at about −20° C. or less for at least 24 hours, at least one week, at least one month, or at least one year. In some aspects, the method further comprises storing the concentrated nucleic acid composition at about −20° C. or less for at least about 24 hours, at least about one week, at least about one month, or at least about one year.

In some aspects, the method further comprises storing the concentrated nucleic acid composition at about 4° C. or less for at least 24 hours, at least one week, at least one month, at least one year, or at least 30 months. In some aspects, the method further comprises storing the concentrated nucleic acid composition at about 4° C. or less for at least about 24 hours, at least about one week, at least about one month, at least about one year, or at least about 30 months.

In some aspects, the concentrated nucleic acid is storage stable. In some aspects, one or more of the following is stable at about 4° C. or less for at least 30 months: particle size, DNA concentration, PPC concentration, PPC/DNA ratio, osmolality, and/or pH.

In some aspects, the method provides for concentrated nucleic acid with improved relative potency of 5× formulation compared to the 1× formulation after 30-month storage at 4° C.

In some aspects, the nucleic acid mixture has a concentration of at least 0.15 mg/mL. In some aspects, the nucleic acid mixture has a concentration of about 0.15 mg/mL to 0.2 mg/mL.

In some aspects, the concentrated nucleic acid composition has a concentration of at least 0.75 mg/mL. In some aspects, the concentration nucleic acid composition has a concentration of about 0.75 mg/mL to 1 mg/mL. In some aspects, the method further comprises (c) lyophilizing the concentrated nucleic acid composition having a concentration of at least 0.75 mg/mL in a volume of 20-40 mL, thereby forming a lyophilized formulation comprising at least 10 mg, at least 15 mg, at least 20 mg, at least 25 mg, or at least 30 mg of the DNA plasmid.

In some aspects, the method further comprises (c) lyophilizing the concentrated nucleic acid composition having a concentration of at least about 0.75 mg/mL in a volume of about 20-40 mL, thereby forming a lyophilized formulation comprising at least about 10 mg, at least about 15 mg, at least about 20 mg, at least about 25 mg, or at least about 30 mg of the DNA plasmid.

In some aspects, the lyophilized formulation comprises 10-50 mg (e.g., about 30 mg) of the DNA plasmid.

In some aspects, the method further comprises (d) reconstituting the lyophilized formulation in a diluent (e.g., water, 5% dextrose, or saline) thereby forming a reconstituted composition.

In some aspects, the nucleic acid mixture comprises GEN-1 nanoparticles.

In some aspects, the ratio of amine nitrogen in the cationic polymer backbone to phosphate in the nucleic is from about 10:1 to about 100:1.

In some aspects, the filler excipient comprises a sugar, a sugar alcohol, a starch, a cellulose, or combinations thereof. In some aspects, the filler excipient comprises one or more of lactose, sucrose, trehalose, dextrose, galactose, mannitol, maltitol, maltose, sorbitol, xylitol, mannose, glucose, fructose, polyvinyl pyrrolidone, glycine, maltodextrin, hydroxymethyl starch, gelatin, sorbitol, ficol, sodium chloride, calcium phosphate, calcium carbonate, and/or polyethylene glycol. In some aspects, the filler excipient comprises lactose.

Certain aspects of the disclosure provides methods whereby low concentration nucleic acid compositions (e.g., 0.15 mg/mL) can be processed using a method comprising a tangential flow filtration (TFF) step to prepare a concentrated (e.g., 0.30 mg/mL to 0.75 mg/mL or higher) nucleic acid composition without affecting the physico-chemical or biological properties of the nucleic acid or nucleic acid composition. In some aspects, nucleic acid composition comprises a DNA plasmid comprising a polynucleotide encoding a therapeutic protein (e.g., a hIL-12 coding nucleic acid) and a lipopolymer (e.g., PPC). In some aspects, the nucleic acid composition comprises GEN-1. In some aspects, GEN-1 can be concentrated according to the methods of the disclosure into a solution comprising 0.30 mg/mL to 0.75 mg/mL. In some aspects, GEN-1 can be concentrated according to the methods of the disclosure into a solution comprising about 0.30 mg/mL to about 0.75 mg/mL.

In some aspects, the concentrated nucleic acid composition (pre-lyophilized) at a concentration (e.g., 0.30 mg/mL to 0.75 mg/mL or higher) can be used directly as a dosing regimen for administration to a subject. In some aspects, the concentrated nucleic acid composition can be stored at −20° C. In some aspects, the concentrated nucleic acid composition (pre-lyophilized) at a concentration (e.g., about 0.30 mg/mL to about 0.75 mg/mL or higher) can be used directly as a dosing regimen for administration to a subject.

In some aspects, nucleic acid composition can be lyophilized according to the disclosure to provide a concentrated lyophilized nucleic acid composition comprising 10 mg to 100 mg (e.g., 10 mg to 100 mg, 10 mg to 90 mg, 10 mg to 80 mg, 10 mg to 70 mg, 10 mg to 60 mg, 10 mg to 50 mg, 10 mg to 40 mg, or 10 mg to 30 mg) of DNA. In some aspects, nucleic acid composition can be lyophilized according to the disclosure to provide a concentrated lyophilized nucleic acid composition comprising 10 mg to 50 mg (e.g., 10 mg to 40 mg, 15 mg to 40 mg, 20 mg to 40 mg, 25 mg to 40 mg, 25 mg to 35 mg, or about 30 mg) of DNA. In some aspects, the concentrated lyophilized nucleic acid composition can be stably stored (e.g., for at 1 year, at least two years, at least three years).

In some aspects, nucleic acid composition can be lyophilized according to the disclosure to provide a concentrated lyophilized nucleic acid composition comprising about 10 mg to about 100 mg (e.g., about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, or about 10 mg to about 30 mg) of DNA. In some aspects, nucleic acid composition can be lyophilized according to the disclosure to provide a concentrated lyophilized nucleic acid composition comprising about 10 mg to about 50 mg (e.g., about 10 mg to about 40 mg, about 15 mg to about 40 mg, about 20 mg to about 40 mg, about 25 mg to about 40 mg, about 25 mg to about 35 mg, or about 30 mg) of DNA. In some aspects, the concentrated lyophilized nucleic acid composition can be stably stored (e.g., for at least about 1 year, at least about two years, at least about three years).

In some aspects, the concentrated lyophilized nucleic acid composition can be reconstituted (e.g., in water, 5% dextrose, or saline). In some aspects, the concentrated nucleic acid compositions allow for a wide range of dosing regimens in vivo after reconstitution of lyophilized composition.

In some aspects, the concentrated lyophilized nucleic acid composition can be reconstituted at 0.5 mg/mL, or 1 mg/mL, or 1.5 mg/mL, 2 mg/mL, or 2.5 mg/mL, or 3 mg/mL, or 3.5 mg/mL, or 4 mg/mL, or 4.5 mg/mL, or 5 mg/mL, or 5.5 mg/mL, or 6 mg/mL, or 6.5 mg/mL, or 7 mg/mL, or 7.5 mg/mL, or 8 mg/mL, or 8.5 mg/mL, or 9 mg/mL, or 9.5 mg/mL, or 10 mg/mL.

In some aspects, the concentrated lyophilized nucleic acid composition can be reconstituted at about 0.5 mg/mL, or about 1 mg/mL, or about 1.5 mg/mL, about 2 mg/mL, or about 2.5 mg/mL, or about 3 mg/mL, or about 3.5 mg/mL, or about 4 mg/mL, or about 4.5 mg/mL, or about 5 mg/mL, or about 5.5 mg/mL, or about 6 mg/mL, or about 6.5 mg/mL, or about 7 mg/mL, or about 7.5 mg/mL, or about 8 mg/mL, or about 8.5 mg/mL, or about 9 mg/mL, or about 9.5 mg/mL, or about 10 mg/mL.

Certain aspects of the disclosure are directed to the concentrated nucleic acid compositions (pre-lyophilization and post-lyophilization) prepared according to the methods disclosed herein.

Certain aspects of the disclosure are directed to a pharmaceutical composition comprising a concentrated nucleic acid compositions (pre-lyophilization and post-lyophilization) prepared according to the methods disclosed herein. In some aspects, the pharmaceutical composition comprises the concentrated nucleic acid composition or reconstituted composition prepared according the methods comprising TFF disclosed herein.

In some aspects, the pharmaceutical composition comprises greater than 6 mg (e.g., 10-100 mg, 10-90 mg, 10-80 mg, 10-70 mg, 10-60 mg, 10-50 mg, 10-40 mg, 20-50 mg, 20-25 mg, 20-40 mg, or 25-35 mg) of DNA plasmid (complexed with PPC) in a volume of less than 70 mL (e.g., 20-70 mL, 20-60 mL, 20-50 mL, or 20-45 mL) of diluent. In some aspects, the pharmaceutical composition comprises greater than 6 mg (e.g., 10-50 mg, 10-40 mg, 20-50 mg, 20-25 mg, 20-40 mg, or 25-35 mg) of DNA plasmid (complexed with PPC) in a volume of less than 50 mL (e.g., 20-45 mL) of diluent. In some aspects, the pharmaceutical composition comprises about 30 mg of DNA plasmid (complexed with PPC) in a volume of 20-45 mL (e.g., about 40 mL) of diluent (e.g., water, 5% dextrose, or saline). In some aspects, the pharmaceutical composition comprises about 150 mg of DNA plasmid (complexed with PPC) in a volume of 200-300 mL (e.g., about 200 mL) of diluent (e.g., water, 5% dextrose, or saline).

In some aspects, the pharmaceutical composition comprises greater than about 6 mg (e.g., about 10-100 mg, about 10-90 mg, about 10-80 mg, about 10-70 mg, about 10-60 mg, about 10-50 mg, about 10-40 mg, about 20-50 mg, about 20-25 mg, about 20-45 mg, about 20-40 mg, or about 25-35 mg) of DNA plasmid (complexed with PPC) in a volume of less than about 70 mL (e.g., about 20-70 mL, about 20-60 mL, about 20-50 mL, or about 20-45 mL) of diluent. In some aspects, the pharmaceutical composition comprises greater than about 6 mg (e.g., about 10-50 mg, about 10-40 mg, about 20-50 mg, about 20-25 mg, about 20-45 mg, about 20-40 mg, or about 25-35 mg) of DNA plasmid (complexed with PPC) in a volume of less than about 50 mL (e.g., about 20-45 mL) of diluent. In some aspects, the pharmaceutical composition comprises about 30 mg of DNA plasmid (complexed with PPC) in a volume of about 20-45 mL (e.g., about 40 mL) of diluent (e.g., water, 5% dextrose, or saline). In some aspects, the pharmaceutical composition comprises about 150 mg of DNA plasmid (complexed with PPC) in a volume of about 200-300 mL (e.g., about 200 mL) of diluent (e.g., water, 5% dextrose, or saline).

In some aspects, the pharmaceutical composition is used to prepare a dosage of 100-200 mg (e.g., 180 mg) of plasmid DNA (complexed with PPC) in less than 300 mL (e.g., 200-300 mL) of diluent (e.g., water, 5% dextrose, or saline). In some aspects, the pharmaceutical composition is used to prepare a dosage of about 100-200 mg (e.g., about 180 mg) of plasmid DNA (complexed with PPC) in less than about 300 mL (e.g., about 200-300 mL) of diluent (e.g., water, 5% dextrose, or saline).

In some aspects, the pharmaceutical composition is in a vial which can hold up to 100 mL, 150 mL, 200 mL, 250 mL or 500 mL (e.g., a 250 mL vial). In some aspects, the vial comprises a volume of 200-300 mL (e.g., about 200 mL) of diluent (e.g., water, 5% dextrose, or saline). In some aspects, the vial comprises 10-50 mg, 10-40 mg, 20-50 mg, 20-25 mg, 20-40 mg, or 25-35 mg (e.g., about 30 mg) of the DNA plasmid (complexed with PPC). In some aspects, the pharmaceutical composition is in a vial which can hold up to about 100 mL, about 150 mL, about 200 mL, about 250 mL or about 500 mL (e.g., a 250 mL vial). In some aspects, the vial comprises a volume of about 200-300 mL (e.g., about 200 mL) of diluent (e.g., water, 5% dextrose, or saline). In some aspects, the vial comprises about 10-50 mg, about 10-40 mg, about 20-50 mg, about 20-25 mg, about 20-45 mg, about 20-40 mg, or about 25-35 mg (e.g., about 30 mg) of the DNA plasmid (complexed with PPC).

In some aspects, the disclosure provides methods for preparing concentrated and stable pharmaceutical compositions. In some aspects, the disclosure is directed to a pharmaceutical composition produced by the methods of the disclosure including at least about 10 mg/mL of a nucleic acid, where the nucleic acid is complexed with a cationic lipopolymer and the complex is suspended in an isotonic solution. The complex suspended in the isotonic solution comprises partially or fully condensed nucleic acid molecules. The cationic lipopolymer comprises a cationic polymer backbone having cholesterol and polyethylene glycol groups (i.e., molecules) covalently attached thereto. The molar ratio of cholesterol molecules to cationic polymer backbone is within a range of from about 0.1 to about 10, and the molar ratio of polyethylene glycol molecules to cationic polymer backbone is within a range of from about 0.1 to about 10. In another aspect, the molar ratio of polyethylene glycol molecules to cationic polymer backbone in the cationic lipopolymer is within a range of from about 1 to about 10. In yet another aspect, the molar ratio of polyethylene glycol molecules to cationic polymer backbone in the cationic lipopolymer is within a range of from about 1 to about 5. In a further aspect, the molar ratio of cholesterol molecules to cationic polymer backbone in the cationic lipopolymer is within a range of from about 0.3 to about 5. In yet a further aspect, the molar ratio of cholesterol molecules to cationic polymer backbone in the cationic lipopolymer is within a range of from about 0.4 to about 1.5.

In some aspects, the composition can further comprise a filler excipient. In some aspects, the filler excipient can include a sugar, a sugar alcohol, a starch, a cellulose, or combinations thereof. In some aspects, the filler excipient is selected from one or more of lactose, sucrose, trehalose, dextrose, galactose, mannitol, maltitol, maltose, sorbitol, xylitol, mannose, glucose, fructose, polyvinyl pyrrolidone, glycine, maltodextrin, hydroxymethyl starch, gelatin, sorbitol, ficol, sodium chloride, calcium phosphate, calcium carbonate, and/or polyethylene glycol. In some aspects, the filler excipient can include lactose, sucrose, trehalose, dextrose, galactose, mannitol, maltitol, maltose, sorbitol, xylitol, mannose, glucose, fructose, polyvinyl pyrrolidone, glycine, maltodextrin, or any combination thereof. In some aspects, the filler excipient comprises or is sucrose. In some aspects, the filler excipient comprises or is lactose.

The resulting composition is suitable for delivery of the nucleic acid to a target cell to elicit, inhibit, or modify a biological response depending on the function of the nucleic acid.

In one aspect, the cholesterol and polyethylene glycol molecules may be independently and directly covalently attached to the cationic polymer backbone. In another aspect, the cholesterol and polyethylene glycol molecules are each covalently attached indirectly to the cationic polymer backbone. For example, the cholesterol molecule may be coupled, directly or indirectly via a linker or spacer, to the polyethylene glycol molecule, which is in turn covalently attached to the cationic polymer backbone. Alternatively, the cholesterol molecule may be directly attached to cationic lipopolymer backbone while the polyethylene glycol molecule is indirectly attached to the lipopolymer via a linker or spacer.

A particular linker between the polyethylene glycol and the cationic polymer backbone is an alkylene group carrying a terminal carboxy group, preferably a straight chain alkylene group of from 1 to 20 carbon atoms, and more preferably from about 2 to about 4 carbon atoms. The terminal carboxy group on the linker, when attached to an amino group of the cationic polymer backbone forms an amide bond between the cationic lipopolymer and the polyethylene glycol. A starting polyethylene glycol suitable for reacting with the cationic polymer backbone molecule is a polyethylene glycol carrying a linker molecule that is terminated by an activating group, e.g., an N-hydroxysuccinimidyl ester. One example of such a polyethylene glycol is methoxypolyethyleneglycol-propionic acid N-hydroxysuccinimidyl ester.

An example of a portion of a cationic lipopolymer structure resulting from the reaction between a polyethyleneimine, cholesteryl chloroformate (stereochemistry omitted), and methoxypolyethyleneglycol-propionic acid N-hydroxysuccinimidyl ester is the following structure. The graphic convention reflects the approximate distribution of primary, secondary and tertiary amino groups in polyethylenimine and, for the purposes of clarity here, assumes a nonexisting regularity of polyethylenimine chain. In some aspects, the gene delivery agent has the following structure:

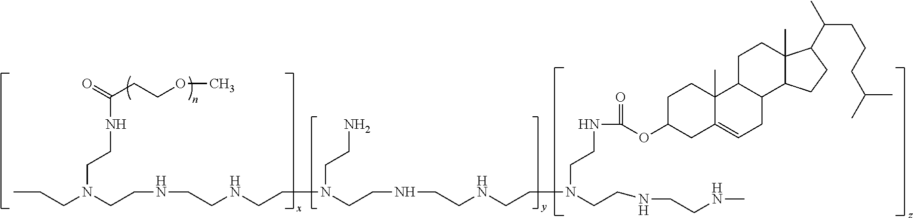

In various aspects of the disclosure, n is typically about 8 to about 20, more particularly about 10 to about 15, and even more particularly about 12; x is typically about 2 to about 3, more particularly about 2.5; y is typically about 6 to about 10, more particularly about 7 to about 9, and even more particularly 7.5; z typically is about 0.4 to about 0.8, more particularly about 0.5 to about 0.7, and even more particularly about 0.6.

Additionally, in some aspects nucleic acids that have previously been condensed using a secondary condensing system may be further condensed using the techniques presented herein to achieve greater stability of nucleic acid at high concentrations. As such, prior to condensation according to aspects of the disclosure, the nucleic acid may be in a partially condensed or a non-condensed form. The secondary condensing system may include any condensing material or technique known to one of ordinary skill in the art, including, but not limited to, cationic lipids, cationic peptides, cyclodextrins, cationized gelatin, dendrimers, chitosan, and combinations thereof.

Various degrees of condensation of a nucleic acid may be achieved for the composition according to aspects of the disclosure. In one aspect, all the nucleic acids or a substantial portion of the nucleic acids in the composition are condensed by forming complexes with the cationic polymer. In another aspect, about 30% by weight of the nucleic acids in the composition are condensed. In yet another aspect, about 50% by weight of the nucleic acid in the composition is condensed. In a further aspect, about 70% by weight of the nucleic acid in the composition is condensed. In yet a further aspect, about 90% by weight of the nucleic acid is condensed. In a further aspect, 90% by weight of the nucleic acid is condensed.

Additionally, the concentration of nucleic acid in the composition will vary depending on the materials used in the composition, the methods of concentration, and the intended use of the nucleic acid. In one aspect, however, the concentration of the nucleic acid is at least about 0.5 mg/mL. In another aspect, the concentration of the nucleic acid is at least about 1 mg/mL. In yet another aspect, the concentration of the nucleic acid is at least about 3 mg/mL. In a further aspect, the concentration of the nucleic acid may be or is at least about 5 mg/mL. In yet a further aspect, the concentration of the nucleic acid may be or is at least about 10 mg/mL. In another aspect, the concentration of the nucleic acid may be or is at least about 20 mg/mL. In yet another aspect, the concentration of the nucleic acid may be or is from about 10 mg/mL to about 40 mg/mL.

Various methods may be utilized to determine the degree of condensation of a nucleic acid composition. For example, in one aspect the composition may be electrophoresed to determine the degree to which nucleic acids in the composition have formed complexes with the cationic polymer added to the composition. The electrostatic attraction of the negatively charged nucleic acid to the positively charged cationic lipopolymer inhibits the nucleic acid from moving through an agarose gel. Accordingly, following electrophoresis, nucleic acids that are condensed by complexing with the cationic polymer remain immobile in the gel, while non-condensed nucleic acids, nucleic acids not associated with the cationic polymer, will have traveled a distance relative to the strength of the electrical current in the gel. In another example, nucleic acid condensation can be determined by particle sizes within the composition. Particle size can be measured by dynamic light scattering. Typically, condensed nucleic acids will have a smaller particle size than non-condensed nucleic acids. Preferred condensed nucleic acids are those in nanoparticles of nucleic acid and cationic lipopolymer having a size of from about 50 nm to about 300 nm, more preferably from about 50-200, and even more preferably from about 50-150 nm.

Any known nucleic acid may be utilized in the compositions and methods according to aspects of the disclosure, including those examples described above. As such, the nucleic acids described herein should not be seen as limiting. In one aspect, for example, the nucleic acid may include a plasmid encoding for a protein, polypeptide, or peptide. Numerous peptides are well known that would prove beneficial when formulated as pharmaceutical compositions according to aspects of the disclosure. In some aspects, the nucleic acid may be a plasmid comprising a nucleic acid coding for interleukin-12.

As has been described, a cationic lipopolymer may include a cationic polymer backbone having cholesterol and polyethylene glycol covalently attached thereto. The cationic polymer backbone may include any cationic polymer known to one of ordinary skill in the art that may be used to condense and concentrate a nucleic acid according to the various aspects of the disclosure. In one aspect, however, the cationic polymer backbone may include polyethylenimine, poly (trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly (lysine), poly (histidine), poly (arginine), cationized gelatin, dendrimers, chitosan, and combinations thereof. In one specific aspect, the cationic polymer backbone may be polyethylenimine.

In a particular aspect, the lipopolymer consists of polyethylenimine (PEI) covalently linked independently to cholesterol and polyethylene glycol. In this aspect, the average PEG:PEI:Cholesterol molar ratio in the cationic lipopolymer is about 1.4-3:1:0.25-1, and preferably about 1.41-2.42:1:0.5-0.9. In a particular aspect, such a lipopolymer has a molecular weight (as the free base) of about from about 3-4 kD, preferably from about 3.25-3.75 kD, and more preferably about 3.54 kD; the corresponding hydrochloric acid salt has a molecular weight of from about 4-5 kD, preferably about 4.5 kD.

Additionally, the molecular weight of a cationic polymer backbone may vary, depending on numerous factors including the properties of the nucleic acid, the intended use of the composition, etc. In one aspect, however, the cationic polymer backbone may have a molecular weight of from about 100 to about 500,000 Daltons. Furthermore, the molecular weight of the other various components of the cationic lipopolymer may also vary. In one aspect, for example, polyethylene glycol may have a molecular weight of from about 50 to about 20,000 Daltons.

In constructing the pharmaceutical compositions of the disclosure, it has been discovered that the molar ratio between the amine nitrogen in the functionalized cationic lipopolymer and the phosphate in the nucleic acid (N:P ratio) may affect the degree to which the nucleic acid may be condensed and/or concentrated. Although the optimal N: P ratio may vary somewhat depending on the chemical characteristics of the nucleic acid, in one aspect the ratio of amine nitrogen in the cationic polymer backbone to phosphate in the nucleic acid is from about 0.1:1 to about 100:1. In another aspect, the ratio of amine nitrogen in the cationic polymer backbone to phosphate in the nucleic acid is from about 3:1 to about 20:1. In yet another aspect, the ratio of amine nitrogen in the cationic polymer backbone to phosphate in the nucleic acid is from about 6:1 to about 15:1. In other aspects, the ratio of amine nitrogen to phosphate in the nucleic acid is from about 3:1 to about 100:1, or about 5:1 to about 100:1, or about 7:1 to about 100:1. In a still another aspect, the ratio is from about 10:1 to about 100:1, or more preferably 10:1 to about 20:1. In one specific aspect, the ratio of amine nitrogen in the cationic polymer backbone to phosphate in the nucleic acid is about 11:1.

It is also contemplated that a filler excipient be included in the pharmaceutical composition. Such filler may provide a variety of beneficial properties to the formulation, such as cryoprotection during lyophilization and reconstitution, binding, isotonic balance, stabilization, etc. It should be understood that the filler material may vary between compositions, and the particular filler used should not be seen as limiting. In one aspect, for example, the filler excipient may include various sugars, sugar alcohols, starches, celluloses, and combinations thereof. In another aspect, the filler excipient may include lactose, sucrose, trehalose, dextrose, galactose, mannitol, maltitol, maltose, sorbitol, xylitol, mannose, glucose, fructose, polyvinyl pyrrolidone, glycine, maltodextrin, hydroxymethyl starch, gelatin, sorbitol, ficol, sodium chloride, calcium phosphate, calcium carbonate, polyethylene glycol, and combinations thereof. In yet another aspect, the filler excipient may include lactose, sucrose, trehalose, dextrose, galactose, mannitol, maltitol, maltose, sorbitol, xylitol, mannose, glucose, fructose, polyvinyl pyrrolidone, glycine, maltodextrin, and combinations thereof. In one aspect, the filler excipient may include sucrose. In another aspect, the filler excipient may include lactose.

The concentration of the filler excipient in the composition may be from about 0.01% to about 10%, more particularly about 0.1% to about 5.0%, and even more particularly from about 1% to about 3%.

In some aspects, it may be beneficial to functionalize the cationic lipopolymer to allow targeting of specific cells or tissues in a subject or culture. Such targeting is well known, and the examples described herein should not be seen as limiting. In one aspect, for example, the cationic lipopolymer may include a targeting moiety covalently attached to either the cationic lipopolymer or to the polyethylene glycol molecule. Such a targeting moiety may allow the cationic lipopolymer to circulate systemically in a subject to locate and specifically target a certain cell type or tissue. Examples of such targeting moieties may include transferrin, asialoglycoprotein, antibodies, antibody fragments, low density lipoproteins, cell receptors, growth factor receptors, cytokine receptors, folate, transferrin, insulin, asialoorosomucoid, mannose-6-phosphate, mannose, interleukins, GM-CSF, G-CSF, M-CSF, stem cell factors, erythropoietin, epidermal growth factor (EGF), insulin, asialoorosomucoid, mannose-6-phosphate, mannose, Lewis^{X} and sialyl Lewis^{X}, N-acetyllactosamine, folate, galactose, lactose, and thrombomodulin, fusogenic agents such as polymixin B and hemaglutinin HA2, lysosomotrophic agents, nucleus localization signals (NLS) such as T-antigen, and combinations thereof. The selection and attachment of a particular targeting moiety is well within the knowledge of one of ordinary skill in the art.

The disclosure also provides lyophilized pharmaceutical compositions that may be stored for long periods of time and reconstituted prior to use. In one aspect, a lyophilized pharmaceutical composition may include a lyophilized mixture of a filler excipient and a nucleic acid condensed with a cationic lipopolymer, where the cationic lipopolymer includes a cationic polymer backbone having cholesterol and polyethylene glycol covalently attached thereto, and wherein the molar ratio of cholesterol to cationic polymer backbone is within a range of from about 0.1 to about 10, and the molar ratio of polyethylene glycol to cationic polymer backbone is within a range of from about 0.1 to about 10. The lyophilized pharmaceutical composition may be in a variety of forms, ranging from dry powders to partially reconstituted mixtures.

The disclosure also includes methods of making various pharmaceutical compositions containing condensed nucleic acids. In one aspect, for example, a method of making a pharmaceutical composition having a condensed nucleic acid concentrated in an isotonic solution to at least 10 mg/mL is provided. Such a method may include mixing a nucleic acid and a cationic lipopolymer in a filler excipient, where the cationic lipopolymer includes a cationic polymer backbone having cholesterol and polyethylene glycol covalently attached thereto, and wherein the molar ratio of cholesterol to cationic polymer backbone is within a range of from about 0.1 to about 10, and the molar ratio of polyethylene glycol to cationic polymer backbone is within a range of from about 0.1 to about 10. The mixture may be lyophilized to a powder to concentrate the nucleic acid mixture and later reconstituted with a diluent to form a solution including at least about 10 mg/mL condensed nucleic acid in an isotonic solution.

One of skill in the art can appreciate that different methods may be used to condensed nucleic acids. Each of these methods, may differ in efficiency, rate of purification, cost, and effort, but are within the knowledge of the skilled artisan. In some aspects of the present disclosure comprises a method of condensing nucleic acids using tangential flow filtration. Tangential flow filtration (TFF) refers to cross-flow filtration wherein the majority of the feed flow travels tangentially across the surface of the filter, rather than into the filter. In some aspects of the present disclosure, TFF is performed before lyophilization of the formulation. In some aspects of the present disclosure, condensed nucleic acid concentrations is at least 0.3 mg/mL, or at least 0.45 mg/mL, or at least 0.6 mg/mL, or at least 0.75 mg/mL. In some aspects of the present disclosure, condensed nucleic acid concentrations is at least about 0.3 mg/mL, at least about 0.45 mg/mL, at least about 0.6 mg/mL, or at least about 0.75 mg/mL.

Condensed nucleic acids percent recovery post-TFF can be calculated by calculating the ratios of pre-TFF cationic lipopolymer/nucleic acid concentrations and post-TFF cationic lipopolymer/nucleic acid concentrations. Condensed nucleic acids percent recovery of the present disclosure is at least 75%, or at least 80%, or at least 85%, or at least 90%, or at least 95%, or at least 100%. Condensed nucleic acids percent recovery of the present disclosure is at least about 75%, or at least about 80%, or at least about 85%, or at least about 90%, or at least about 95%, or about 100%.

In some aspects of the present disclosure, the condensed nucleic acid post-TFF is frozen and stored at about −20° C. In some embodiments, the condensed nucleic acid post-TFF is frozen and stored at about −30° C., about −40° C., about −50° C., about −60° C., about −70° C., or about −80° C. In some embodiments, the condensed nucleic acid post-TFF is flash frozen. In some embodiments, the condensed nucleic acid post-TFF is flash frozen using dry ice or liquid nitrogen.

In some aspects of the present disclosure, the condensed nucleic acid is frozen for greater than one week, greater than 1 month, greater than 3 months, greater than 6 months, greater than 9 months, greater than 12 months, greater than 15 months, greater than 18 months, or greater than 24 months, after which the condensed nucleic acid that has been frozen is thawed and has same or substantially the same assay performance as condensed nucleic acid that had not been frozen. In some aspects of the present disclosure, the condensed nucleic acid is frozen for greater than about one week, greater than about 1 month, greater than about 3 months, greater than about 6 months, greater than about 9 months, greater than about 12 months, greater than about 15 months, greater than about 18 months, or greater than about 24 months, after which the condensed nucleic acid that has been frozen is thawed and has same or substantially the same assay performance as condensed nucleic acid that had not been frozen.

Generally, the composition may be obtained by mixing a nucleic acid solution with a cationic lipopolymer solution in the presence of a disaccharide sugar followed by lyophilization and reconstitution in an isotonic solution. This process is scalable, producing a few milligrams (bench scale) to several thousand milligrams (GMP scale) of the highly concentrated nucleic acid formulations with prolonged shelf life. As has been described, the cationic lipopolymer has a cationic polymer backbone to which polyethylene glycol and cholesterol are attached by covalent linkages. In the case of a polyethylenimine backbone, in one aspect the stoichiometry between polyethylene glycol and polyethylenimine and between cholesterol and polyethylenimine is in the range of 0.5-10 and 0.1-10, respectively. In the case of a polyethylenimine backbone, in one aspect the stoichiometry between polyethylene glycol and polyethylenimine and between cholesterol and polyethylenimine is in the range of about 0.5-10 and about 0.1-10, respectively. The chemical composition of the cationic polymer may be important to obtaining highly concentrated stable nucleic acid formulations. Cationic polymers that do not exhibit cholesterol and PEG attachment do not tend to produce stable highly concentrated formulations, as is shown in the Examples below.

The compositions according to aspects of the disclosure can also be combined with other condensed complexes of nucleic acid to achieve greater stability of the complexes at high nucleic acid concentrations. For example, various amounts of PEG-PEI-Cholesterol can be added to enhance the stability of other nucleic acid delivery systems that are generally unstable at high nucleic acid concentrations.

In various aspects, the synthetic delivery systems include a nucleic acid and cationic carrier which may be prepared by various techniques available in the art. A number of cationic carriers for nucleic acids are known: for example, polyethylenimine, poly (trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly (lysine), poly (histidine), poly (arginine), cationized gelatin, dendrimers, chitosan, cationic lipids such as 1,2-Dioleoyl-3-Trimethylammonium-Propane (DOTAP), N-[1-(2,3-dioleoyloxy) propyl]-N,N,N-trimethylammonium chloride (DOTMA), 1-[2-(oleoyloxy)ethyl]-2-oleyl-3-(2-hydroxyethyl) imidazolinium chloride (DOTIM), 2,3-dioleyloxy-N-[2 (sperminecarboxamido)ethyl]-N,N-dimethyl-1-propanaminium trifluoroacetate (DOSPA), 3ß-[N-(N′,N′-Dimethylaminoethane)-carbamoyl] Cholesterol Hydrochloride (DC-Cholesterol HCl) diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride DODAC) and combinations thereof. When these delivery systems are combined with PEG-PEI-Cholesterol, stability of the nucleic acid delivery system is increased.

Condensed nucleic acids percent biological activity post-TFF can be calculated by calculating the biological activity of pre-TFF cationic nucleic acid concentrations and the biological activity of post-TFF condensed nucleic acids. Percent biological activity of the present disclosure is at least 75%, or at least 80%, or at least 85%, or at least 90%, or at least 95%, or at least 100%. Percent biological activity of the present disclosure is at least about 75%, at least about 80%, at east about 85%, at least about 90%, at least about 95%, or about 100%.

Lyophilized formulations at higher concentrations prepared by the methods disclosed herein are stable (e.g., the particle size and osmolality).

### III. Gene Delivery Polymer

Certain aspects of the disclosure relate to methods for preparation and use of concentrated nucleic acid formulations comprising a gene delivery polymer disclosed herein.

In some aspects, the lipopolymer comprises of polyethyleneimine (PEI) covalently linked independently to cholesterol and polyethylene glycol (PEG) groups.

The present disclosure provides a polymeric system, PEG-PEI-Cholesterol (PPC), which differs from WSLP (PEI-Cholesterol) in that it contains PEG moieties and yields significantly higher transfection efficiency in tumors. The addition of PEG is designed to enhance the stability of the nucleic acid/polymer complexes in the biological milieu to circumvent for this deficiency in the prior art (WSLP). Furthermore, the addition of PEG chains allows for the incorporation of ligands on to the PPC chain to improve the tissue selectivity of delivery. For example, the cholesterol moiety which is directly linked to the PEI back bone in the prior art (WSLP) may be extended farther from the PEI backbone to create a more flexible geometry for cell receptor interaction. Controlling the number of PEG molecules per unit of the PEI backbone is important to achieve optimal enhancement in transfection activity. A preferred range of composition was a PEG: PEI molar ratio of 2-4 at a fixed cholesterol content. In some aspects, the optimal ratio between PEI and cholesterol was 1:0.5 to 1:1. In some aspects, the optimal ratio between PEI and cholesterol was about 1:0.5 to 1:1.

In some aspects, the polynucleotide encodes human IL-12.

In some aspects, nucleic acid vector (e.g., a plasmid) comprises a promoter operably linked to a nucleic acid encoding a p35 subunit of IL-12 and a promoter operably linked to a nucleic acid encoding a p40 subunit of IL12.

In some aspects, nucleic acid vector (e.g., a plasmid) comprises an intron, a 3′UTR (e.g., hGH 3′UTR), an antibiotic resistance gene, or any combination thereof.

In some aspects, the lipopolymer comprises a polyethyleneimine (PEI) covalently linked independently to cholesterol and polyethylene glycol (PEG) groups.

In some aspects, the nanoparticle disclosed herein comprises a DNA plasmid that encodes human IL-12.

In some aspects, wherein nanoparticle comprises a synthetic polymer facilitating plasmid delivery that is a lipopolymer.

In some aspects, the lipopolymer further comprises polyethyleneimine (PEI) covalently linked independently to cholesterol and polyethylene glycol (PEG) groups.

In some aspects, the gene delivery polymer is a cationic polymer or a non-condensing polymer. The cationic polymer is selected from the group comprising polylysine, polyethylenimine, functionalized derivatives of polyethylenimine (PEI), polypropylenimine, aminoglycoside-polyamine, dideoxy-diamino-b-cyclodextrin, spermine and spermidine. One example of a cationic gene delivery polymer suitable for the present disclosure is a PEI derivative comprising a PEI backbone, a lipid, and a hydrophilic polymer spacer wherein the lipid is directly bound to the polyethylenimine backbone or covalently bound to the polyethylene glycol spacer, which in turn is bound, via a bio compatible bond, to the PEI.

The cationic gene delivery polymer of the present disclosure may further comprise a targeting moiety including antibodies or antibody fragments, cell receptors, growth factor receptors, cytokine receptors, folate, transferrin, epidermal growth factor (EGF), insulin, asialoorosomucoid, mannose-6-phosphate (monocytes), mannose (macrophage, some B cells), Lewis× and sialyl Lewis× (endothelial cells), N-acetyllactosamine (T cells), galactose (colon carcinoma cells), and thrombomodulin (mouse lung endothelial cells), fusogenic agents such as polymixin B and hemaglutinin HA2, lysosomotrophic agents, nucleus localization signals (NLS) such as T-antigen, and the like. Another gene delivery polymer is a non-condensing polymer selected from the group comprising polyvinylpyrrolidone, polyvinylalcohol, poly (lactide-co-glycolide) (PLGA) and triblock copolymers of PLGA and PEG. The gene delivery polymer may also be a non-condensing polymer. Examples of such non-condensing polymers include polyvinyl pyrollidone, polyvinyl alcohol, poloxamers, polyglutamate, gelatin, polyphosphoesters, silk-elastin-like hydrogels, agarose hydrogels, lipid microtubules, poly (lactide-co-glycolide) and polyethyleneglycol-linked poly (lactide-co-glycolide).

The gene delivery polymer is a cationic polymer or a non-condensing polymer. The cationic polymer is selected from the group comprising polylysine, polyethylenimine, functionalized derivatives of polyethylenimine, polypropylenimine, aminoglycoside-polyamine, dideoxy-diamino-b-cyclodextrin, spermine and spermidine. One example of a cationic gene delivery polymer suitable for the present invention is a polyethylenimine derivative comprising a polyethylenimine (PEI) backbone, a lipid, and a polyethylene glycol spacer wherein the lipid is directly bound to the polyethylenimine backbone or covalently bound to the polyethylene glycol spacer, which in turn is bound, via a biocompatible bond, to the PEI.

In some aspects, the gene delivery polymer comprises a lipopolyamine with the following formula:

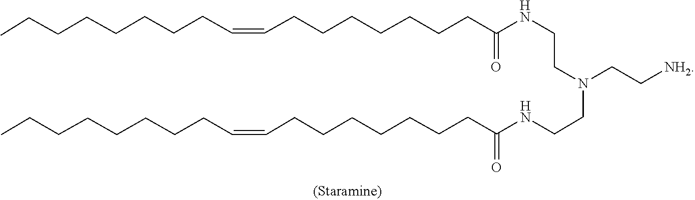 [SMILES: CCCCCCCC/C=C\CCCCCCCC(=O)NCCN(CCN)CCNC(=O)CCCCCCC/C=C\CCCCCCCC]

In some aspects, the gene delivery polymer comprises a mixture of the lipopolyamine and an alkylated derivative of the lipopolyamine. In some aspects, the alkylated derivative of the lipopolyamine is a polyoxyalkylene, polyvinylpyrrolidone, polyacrylamide, polydimethylacrylamide, polyvinyl alcohol, dextran, poly (L-glutamic acid), styrene maleic anhydride, poly-N-(2-hydroxypropyl) methacrylamide, or polydivinylether maleic anhydride. In some aspects, the alkylated derivative of the lipopolyamine has the following formula:

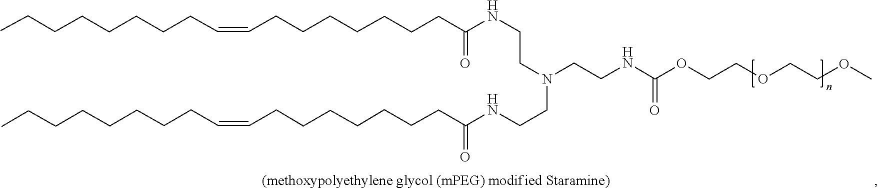

wherein n represents an integer from 10 to 100 repeating units containing 2-5 carbon atoms each. In some aspects, the alkylated derivative of the lipopolyamine has the following formula:

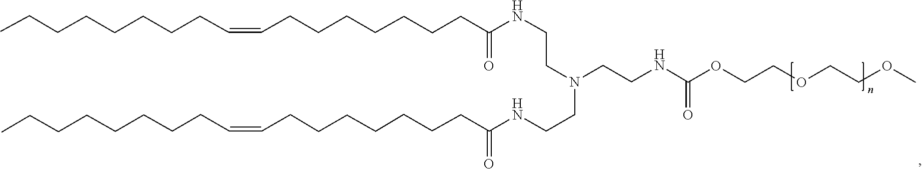

wherein n=11 (Staramine-mPEG515). In some aspects, the alkylated derivative of the lipopolyamine has the following formula:

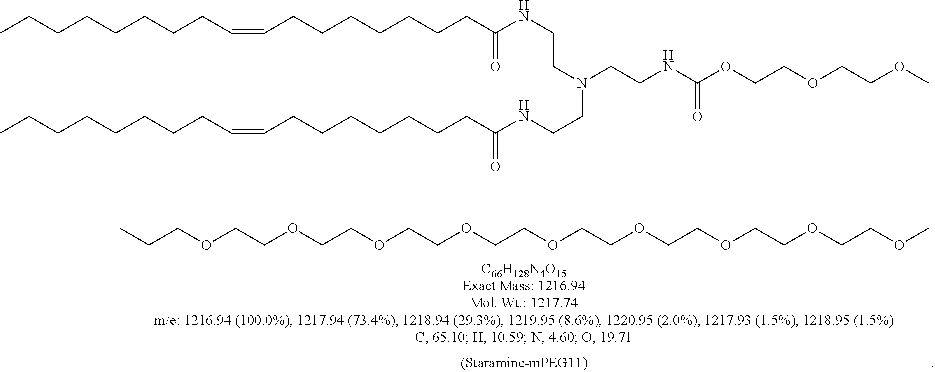

In some aspects, the ratio of the lipopolyamine to the alkylated derivative of the lipopolyamine in the mixture is 1:1 to 10:1. In some aspects, the lipopolyamine is present in an amount sufficient to produce a ratio of amine nitrogen in the lipopolyamine to phosphate in the nucleic acid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the lipopolyamine to phosphate in the nucleic acid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the lipopolyamine to phosphate in the nucleic acid vector is from about 1:10 to about 10:1.

In some aspects, the gene delivery polymer comprises a lipopolyamine with the following formula:

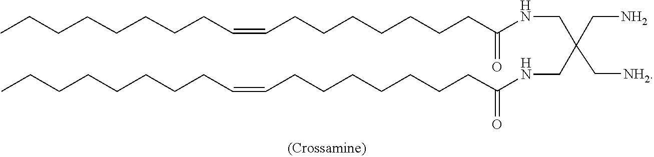 [SMILES: CCCCCCCC/C=C\CCCCCCCC(=O)NCC(CN)(CN)CNC(=O)CCCCCCC/C=C\CCCCCCCC]

In some aspects, the gene delivery polymer comprises a mixture of the lipopolyamine and an alkylated derivative of the lipopolyamine. In some aspects, the alkylated derivative of the lipopolyamine is a polyoxyalkylene, polyvinylpyrrolidone, polyacrylamide, polydimethylacrylamide, polyvinyl alcohol, dextran, poly (L-glutamic acid), styrene maleic anhydride, poly-N-(2-hydroxypropyl) methacrylamide, or polydivinylether maleic anhydride. In some aspects, the ratio of the lipopolyamine to the alkylated derivative of the lipopolyamine in the mixture is 1:1 to 10:1. In some aspects, the ratio of the lipopolyamine to the alkylated derivative of the lipopolyamine in the mixture is about 1:1 to 10:1. In some aspects, the lipopolyamine is present in an amount sufficient to produce a ratio of amine nitrogen in the lipopolyamine to phosphate in the nucleic acid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the lipopolyamine to phosphate in the nucleic acid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the lipopolyamine to phosphate in the nucleic acid vector is from about 1:10 to about 10:1.

In some aspects, the gene delivery polymer comprises a poloxamer back-bone having a metal chelator covalently coupled to at least one terminal end of the poloxamer backbone. In some aspects, the metal chelator is coupled to at least two terminal ends of the poloxamer backbone. In some aspects, the poloxamer backbone is a poloxamer backbone disclosed in U.S. Publ. No. 2010/0004313, which is herein incorporated by reference in its entirety. In some aspects, the metal chelator is a metal chelator disclosed in U.S. Publ. No. 2010/0004313. In some aspects, the gene delivery polymer comprises a polymer having the following formula:

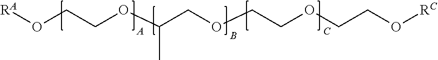

and pharmaceutically acceptable salts thereof, wherein:
A represents an integer from 2 to 141;
B represents an integer from 16 to 67;
C represents an integer from 2 to 141;
RA and RC are the same or different, and are R′-L- or H, wherein at least one of RA and RC is R′-L-;
L is a bond, —CO—, —CH2-O—, or —O—CO—; and
R′ is a metal chelator.

In some aspects, the metal chelator is RNNH—, RN2N—, or (R″—(N(R″)—CH2CH2)x)2-N—CH2CO—, wherein each x is independently 0-2, and wherein R″ is HO2C—CH2-. In some aspects, the metal chelator is a crown ether selected from the group consisting of 12-crown-4, 15-crown-5, 18-crown-6, 20-crown-6, 21-crown-7, and 24-crown-8. In some aspects, the crown ether is a substituted-crown ether, wherein the substituted crown ether has:
(1) one or more of the crown ether oxygens independently replaced by NH or S,
(2) one or more of the crown ether —CH2-CH2-moieties replaced by —C6H4-, —C10H6-, or —C6H10-,
(3) one or more of the crown ether —CH2-O—CH2-moieties replaced by —C4H20- or —C5H3N—, or
(4) any combination thereof.

In some aspects, the metal chelator is a cryptand, wherein the cryptand is selected from the group consisting of (1,2,2) cryptand, (2,2,2) cryptand, (2,2,3) cryptand, and (2,3,3) cryptand. In some aspects, the cryptand is a substituted-cryptand, wherein the substituted cryptand has:
(1) one or more of the crypthand ether oxygens independently replaced by NH or S,
(2) one or more of the crown ether —CH2-CH2-moieties replaced by —C6H4-, —C10H6-, or —C6H10-,
(3) one or more of the crown ether —CH2-O—CH2-moieties replaced by —C4H2O— or —C5H3N—, or
(4) any combination thereof.

In some aspects, the gene delivery polymer is Crown Poloxamer (aza-crown-linked poloxamer), wherein the Crown Poloxamer comprises a polymer having a formula disclosed herein. In some aspects, the gene delivery polymer is Crown Poloxamer (aza-crown-linked poloxamer), wherein the gene delivery polymer has the following formula:

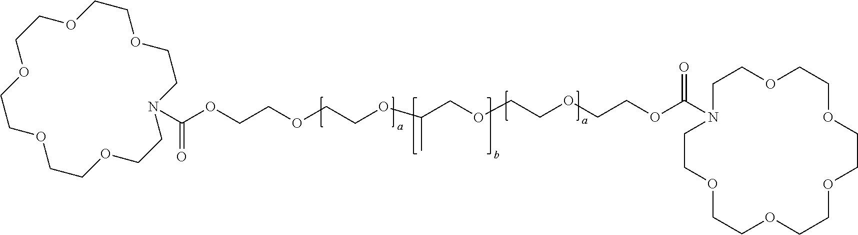

or pharmaceutically acceptable salts thereof, wherein:
a represents an integer of about 10 units; and
b represents an integer of about 21 units; and
wherein the total molecular weight of the polymer is about 2,000 Da to about 2,200 Da.

In some aspects, the crown poloxamer is present in a solution with the nucleic acid vector from about 0.1%-about 5% or about 0.5%-about 5%.

In some aspects, the gene delivery polymer is present in a solution with the nucleic acid vector from about 0.1%-about 5% or about 0.5%-about 5%.

In some aspects, the gene delivery polymer is a β-amino ester. In some aspects, the polymer is present in a solution with the nucleic acid vector from about 0.1%-about 5% or about 0.5%-about 5%.

In some aspects, the gene delivery polymer is a poly-inosinic-polycytidylic acid. In some aspects, the poly-inosinic-polycytidylic acid is present in a solution with the nucleic acid vector from about 0.1%-about 5% or about 0.5%-about 5%.

In some aspects, the gene delivery polymer further comprises benzalkonium chloride.

In some aspects, the gene delivery polymer comprises BD15-12. In some aspects, the ratio of nucleotide to BD15-12 polymer (N:P) is 5:1.

In some aspects, the gene delivery polymer comprises Omnifect. In some aspects, the ratio of nucleotide to Omnifect polymer (N:P) is 10:1.

In some aspects, the gene delivery polymer comprises Crown Poloxamer (aza-crown-linked poloxamer). In some aspects, the ratio of nucleotide to Crown Poloxamer (N:P) is 5:1. In some aspects, the gene delivery polymer comprises Crown Poloxamer and a PEG-PEI-cholesterol (PPC) lipopolymer. In some aspects, the gene delivery polymer comprises Crown Poloxamer and benzalkonium chloride. In some aspects, the gene delivery polymer comprises Crown Poloxamer and Omnifect. In some aspects, the gene delivery polymer comprises Crown Poloxamer and a linear polyethyleneimine (LPEI). In some aspects, the gene delivery polymer comprises Crown Poloxamer and BD15-12.

In some aspects, the gene delivery polymer comprises Staramine and mPEG modified Staramine. In some aspects, the mPEG modified Staramine is Staramine-mPEG515. In some aspects, the mPEG modified Staramine is Staramine-mPEG11. In some aspects, the ratio of Staramine to mPEG modified Staramine is 10:1. In some aspects, the nucleotide to polymer (N:P) ratio is 5:1. In some aspects, the gene delivery polymer comprises Staramine, mPEG modified Staramine, and Crown Poloxamer. In some aspects, the gene delivery polymer comprises Staramine, Staramine-mPEG515, and Crown Poloxamer. In some aspects, the gene delivery polymer comprises Staramine, Staramine-mPEG11, and Crown Poloxamer.

In some aspects, the gene delivery polymer comprises a poloxamer backbone disclosed in WO 2022/072910 A1, which is herein incorporated by reference in its entirety.

In some aspects, the nanoparticle that comprises a DNA plasmid that encodes interleukin-12 (IL-12) and a synthetic gene delivery polymer (e.g., PPC) facilitating plasmid delivery, e.g., intraperitoneally.

### IV. GEN-1 Formulations

GEN-1 is a gene-based immunotherapy, comprising a human IL-12 gene expression plasmid and a synthetic lipopolymer delivery system (Thaker, Premal H. et al., Future Oncol. (2019) 15 (4), 421-438). GEN-1 includes a plasmid vector encoding the p35 and p40 subunits of human IL-12 genes, each under the control of a cytomegalovirus (CMV) promotor, and a synthetic lipopolymer delivery system, polyethylene glycol (PEG)-polyethylenimine (PEI)-cholesterol (PPC). In some aspects, GEN-1 is formulated as a lyophilized powder that can be reconstituted at the bedside, e.g., at doses up to 0.5 mg/mL. In some aspects, the GEN-1 DNA plasmid is delivered using a synthetic polymer facilitating plasmid delivery that is a lipopolymer.

In some aspects, the GEN-1 formulation is prepared at a dosage form of about 35 mg/m² to about 120 mg/m². In some aspects, the GEN-1 formulation prepared at a dosage form of about 35 mg/m² to about 110 mg/m². In some aspects, the GEN-1 formulation is prepared at a dosage form of about 50 mg/m² to about 100 mg/m². In some aspects, the GEN-1 formulation is prepared at a dosage form of about 100 mg/m². In some aspects, the GEN-1 formulation is prepared at a dosage form of about 60 mg/m².

In some aspects, the GEN-1 formulation prepared at a dosage form of about 100 mg/m² comprising about 180 mg of DNA (plasmid comprising coding sequence for hIL-12).

The present disclosure provides methods for preparing a GEN-1 formulation that is at least 2×, at least 3×, at least 4×, at least 5×, at least 6×, at least 7×, at least 8×, at least 9×, or at least 10× more concentrated than GEN-1 formulations prepared using methods known in the art. For example, using known methods a dose comprising about 180 mg DNA requires about 30 vials (50 mL vials) to reconstitute (i.e., each vial carrying about 6 mg DNA to reconstitute). This is a commercially challenging preparation. The methods of the current disclosure allow for DNA preparations comprising about 5× more material per vial. For a dose comprising about 180 mg, the method disclosed herein reduces the number of vials from about 30 vials to about 6 vials (50 mL vials) to reconstitute (i.e., each vial carrying about 30 mg DNA to reconstitute). In some aspects, for a dose comprising about 180 mg of plasmid DNA (GEN-1), the method disclosed herein reduces the number of vials from about 30 vials to about 2 vials (250 mL vials) to be reconstituted (e.g., each vial carrying about 150 mg DNA to reconstitute).

In some aspects, the GEN-1 lyophilized formulations at higher concentrations prepared by the methods disclosed herein are stable (e.g., the particle size and osmolality).

### V. Methods of Use

Certain aspects of the disclosure are directed to methods of using the concentrated nucleic acid compositions prepared according the methods disclosed herein. For example, in one aspect a method of transfecting a mammalian cell may include contacting the mammalian cell with a composition as described herein, and incubating the mammalian cell under conditions to allow the composition to enter the cell and elicit biological activity of the nucleic acid. Such transfection techniques are known to those of ordinary skill in the art. Additionally, in another aspect a targeted tissue may be transfected by delivering the composition into a warm blooded organism or subject. Such delivery may be by a form of administration such as intratumoral, intraperitoneal, intravesicle, intravenous, intra-arterial, intratracheal, intrahepaticportal, oral, intracranial, intramuscular, intraarticular and combinations thereof. Such targeted tissue may include any tissue or subset of tissue that would benefit from transfection. For example, and without limitation, such targeted tissue may include ovary, uterus, stomach, colon, rectum, bone, blood, intestine, pancreas, breast, head, neck, lungs, spleen, liver, kidney, brain, thyroid, prostate, urinary bladder, thyroid, skin, abdominal cavity, thoracic cavity, and combinations thereof.

The present disclosure provides methods for treatment of mammalian cancer or hyperproliferative disorders by intratumoral, intraperitoneal, intravesicle, intravenously, intravesicularly, intratracheal, intracranial or systemic administration of pharmaceutical compositions comprising a plasmid-based gene expression system and a gene delivery polymer, without a chemotherapeutic drug. The mammalian cancer is selected from a group consisting of primary or metastasized tumors of the ovary. In some aspects, the nucleic acid is a plasmid-based gene expression system containing a DNA sequence which encodes human interleukin-12 (e.g., GEN-1).

The treatment of tumors with the said pharmaceutical composition (e.g., GEN-1) prepared according the methods disclosed herein results in tumor shrinkage and extension of life span. In some aspect, the method of treatment comprises a combination therapy.

In some aspects, GEN-1 can be delivered intraperitoneally (i.p.) to produce local and persistent levels of IL-12 at the tumor site in patients with advanced ovarian cancer. GEN-1 can be administered alone or in combination with chemotherapy.

In some aspects, the combination therapy comprises use of a nucleic acid composition (e.g., GEN-1) prepared according to the methods disclosed herein and a chemotherapy (chemotherapeutic agents). The efficacy of the methods of this disclosure can be defined as, but not limited to, shrinkage in tumor size or reduction in tumor density, an increase in lymphocyte count or increase in neutrophil count or improvement in survival, or all of the above. In some aspects, the combination of GEN-1 with chemotherapy (chemotherapeutic agents) according to the methods of the present invention can lower the toxicity of the chemotherapeutic agent and reverses tumor resistance to chemotherapy. The toxicity herein is defined as any treatment related adverse effects on clinical observation including but not limited to abnormal hematology or serum chemistry or organ toxicity. Furthermore, the combination of GEN-1 with a suboptimal dose of chemotherapy (chemotherapeutic agents) according to the method of the present invention enhances the anticancer effect to a level equal to or higher than that of achieved with the optimal dose of the chemotherapeutic agent but with lesser toxicity.

In some aspects, the nucleic acid composition (e.g., GEN-1) prepared according to the methods disclosed herein is administered at a dose of about 35 mg/m² to about 120 mg/m². In some aspects, the nucleic acid composition (e.g., GEN-1) prepared according to the methods disclosed herein is administered at a dose of about 35 mg/m² to about 110 mg/m². In some aspects, the nucleic acid composition (e.g., GEN-1) prepared according to the methods disclosed herein is administered at a dose of about 50 mg/m² to about 100 mg/m². In some aspects, the nucleic acid composition (e.g., GEN-1) prepared according to the methods disclosed herein is administered at a dose of about 100 mg/m². In some aspects, the nucleic acid composition (e.g., GEN-1) prepared according to the methods disclosed herein is administered at a dose of about 60 mg/m².

In some aspects, the GEN-1 formulation prepared according to the methods disclosed herein is administered at a dose of about 100 mg/m² comprising about 180 mg of DNA (plasmid comprising coding sequence for hIL-12). In some aspects, the dose is administered to an average 1.8 m² surface area of the subject.

The present disclosure provides methods for preparing a GEN-1 formulation that is at least 2×, at least 3×, at least 4×, at least 5×, at least 6×, at least 7×, at least 8×, at least 9×, or at least 10× more concentrated than GEN-1 formulations prepared using methods known in the art. For example, using known methods a dose comprising about 180 mg DNA requires about 30 vials (50 mL vials) to reconstitute (i.e., each vial carrying about 6 mg DNA to reconstitute). This is a commercially challenging preparation. The methods of the current disclosure allow for DNA preparations comprising about 5× more material per vial. For a dose comprising about 180 mg, the method disclosed herein reduces the number of vials from about 30 vials to about 6 vials (50 mL vials) to reconstitute (i.e., each vial carrying about 30 mg DNA to reconstitute). In some aspects, for a dose comprising about 180 mg of plasmid DNA (GEN-1), the method disclosed herein reduces the number of vials from about 30 vials to about 2 vials (250 ml vials) to reconstitute (i.e., each vial carrying about 150 mg DNA to reconstitute).

Certain aspects of the disclosure are related to a combination therapy comprising: (i) a nucleic acid vector (e.g., a plasmid) comprising a polynucleotide formulated with a lipopolymer (e.g., a nanoparticle) which is prepared according to the methods disclosed herein; and (ii) an anticancer agent. In some aspects, the polynucleotide encodes human interleukin-12 (hIL-12).

In some aspects, nucleic acid vector (e.g., a plasmid) comprises a promoter operably linked to a nucleic acid encoding a p35 subunit of IL-12 and a promoter operably linked to a nucleic acid encoding a p40 subunit of IL-12.

In some aspects, nucleic acid vector (e.g., a plasmid) comprises an intron, a 3′UTR (e.g., hGH 3′UTR), an antibiotic resistance gene, or any combination thereof.

In some aspects, the lipopolymer comprises a polyethyleneimine (PEI) covalently linked independently to cholesterol and polyethylene glycol (PEG) groups.

In some aspects, the anticancer agent is a chemotherapeutic agent.

In some aspects, the chemotherapeutic agent is selected from a group consisting of topoisomerase inhibitors (e.g., irinotecan, topotecan, doxorubicin, epirubicin, idarubicin), anti-microtubule agents (e.g., paclitaxel, docetaxel), alkylating agents (e.g., cyclophosphamide, dacarbizine), platinum-based drugs (cisplatin, carboplatin, oxaliplatin), anti-metabolites (e.g., gemcitabine, methotrexate, 5-fluorouracil), or combinations thereof.

In some aspects, the chemotherapeutic agent is selected from the group consisting of doxorubicin, paclitaxel, carboplatin, docetaxel, nab-paclitaxel, olaparib, and any combination thereof.

In some aspects, the anticancer agent is doxorubicin. In some aspects, the anticancer agent is paclitaxel. In some aspects, the anticancer agent is carboplatin. In some aspects, the anticancer agent is docetaxel. In some aspects, the anticancer agent is nab-paclitaxel. In some aspects, the anticancer agent is olaparib.

In some aspects, the nucleic acid vector formulated with the lipopolymer is administered prior to, concurrently with, or after the anticancer agent.

In some aspect, the method of treatment further comprises a surgery to remove all or part of a tissue or tumor (e.g., interval cytoreductive surgery) in the subject.

In some aspects, the nucleic acid vector formulated with the lipopolymer is administered intratumorally or intraperitoneally.

In some aspects, the nucleic acid vector formulated with the lipopolymer is administered intravenously.

In some aspects, a surgery to remove all or part of a tissue or tumor is performed (e.g., cytoreductive surgery) (e.g., first), followed by the administration of an anticancer agent, followed by administration of the nucleic acid vector formulated with the lipopolymer prepared according to the methods disclosed herein.

### EXAMPLES

The following examples are provided to promote a more clear understanding of certain embodiments of the invention, and are in no way meant as a limitation thereon.

### Example 1: Small-Scale Preparation of Liquid Formulations of Condensed Nucleic Acid with a Cationic Lipopolymer

This example illustrates preparation of formulations of fully condensed nucleic acid at bench-scale production. This involved preparation of nucleic acid complexes with a cationic polymer followed by lyophilization and reconstitution to isotonic solutions. The nucleic acids used included a plasmid DNA encoding for IL-12 or luciferase gene, where the polymer comprised a polyethylenimine (PEI) backbone covalently linked to polyethylene glycol (PEG) and cholesterol (Chol) (PEG-PEI-Chol, also referred to as PPC). The molar ratio between PEG and PEI and between cholesterol and PEI was 0.5-10 and 0.1-10, respectively. First, the DNA and PPC solutions were separately prepared at 5 mg/mL in water for injection and subsequently diluted to 0.15 mg/mL (DNA) and 0.554 mg/mL (PPC) at 3% lactose. The DNA in lactose solution was added to the PPC in lactose solution using a micropipette to a nitrogen to phosphate ratio (N:P ratio) of 11:1, and the formulation was incubated for 15 minutes at room temperature to allow the complexes to form.

The PPC/DNA complexes in 3% lactose were lyophilized using a FREEZONE freeze dry System from LABCONCO Corp. Kansas City, MO. 500 μl of prepared formulation was added to 2 mL borosilicate glass vials, which were then lyophilized using a freeze drying program including of the following segments: (1) freezing segment (Ramp 0.25° C./min, hold at 34° C. for 4 hrs), (2) primary drying segment (hold at 34° C. for 24 hrs), (3) secondary drying segment (Ramp to 20° C. and hold for 24 hrs), and (4) Ramp to 4° C. at 0.25° C./min.

The resultant lyophilized powder was reconstituted with water for injection to various concentrations ranging from 0.1 mg/mL to 20 mg/mL DNA. A typical batch of small-scale preparation amounted to 100-200 mg of fully formulated DNA.

### Example 2: Scaled-Up Preparation of Liquid Formulations of Condensed Nucleic Acid with a Cationic Lipopolymer

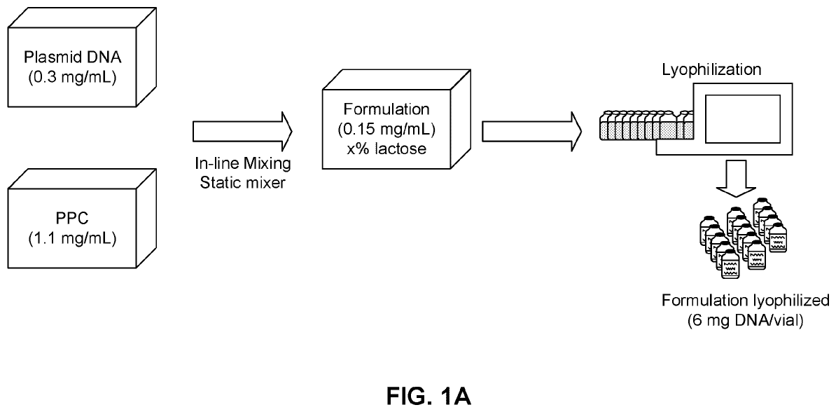

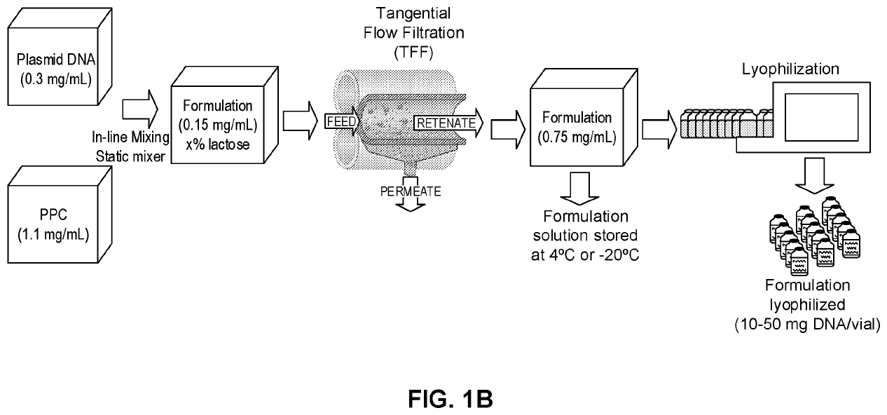

This example illustrates a preparation of formulations of condensed nucleic acid to produce up to 6 mg DNA/vial (e.g., 100 mL vials), as is shown in FIG. 1A. This process was able to produce at least 6000 mg of fully formulated DNA (as compared to 100-200 mg DNA produced from the small-scale preparation described in Example 1) and can be expanded to even higher production amounts. The scaled-up method involved mixing of the bulk DNA and polymer solutions with a peristaltic pump achieving an online mixing scenario to form the complexes followed by freeze-drying cycles compatible for large load. Briefly, the DNA and PPC solutions were prepared at 0.3 mg/mL and 1.1 mg/mL in 3% lactose, respectively. The two components are combined at a constant flow rate using a peristaltic pump (WATSON MARLOW, SCI 400) with a 0.89 mm internal diameter of silicon tubing (WATSON MARLOW, Z982-0088) at a flow rate of 225±25 mL/min. The two mixtures were joined by a polypropylene T-connector at the end of each tube. Mixing polymer and DNA solutions resulted in instant formation of nanoparticles at a concentration of 0.15 mg/mL formulation. Forty milliliters of the formulated complexes are placed in 100 mL glass vials and lyophilized using a freeze-drying program consisting of the following segments: (1) pre-freeze at −50 C for up to 720 minutes, (2) primary drying at −40 C for up to 180 minutes and then at −34 C for up to 1980 minutes at 65 μm Hg, and (3) secondary drying at −25 C for up to 720 minutes, —15 C for up to 3180 minutes, —10 C for up to 1500 minutes, and 4 C for up to 1440 minutes at 65 μm Hg.

Each 100 mL glass vial contained 6 mg of lyophilized DNA powder. The resultant lyophilized DNA powder was reconstituted with water for injection to various concentrations ranging from 0.1 mg/mL to 20 mg/mL DNA. A typical batch of this scale amounts to about 6000 mg of fully formulated DNA.

### Example 3: Preparation of Concentrated Liquid Formulations of Condensed Nucleic Acid with a Cationic Lipopolymer Using Tangential Flow Filtration (TFF)

This example illustrates a preparation of highly concentrated formulations of condensed nucleic acid to produce up to 30 mg DNA/vial (e.g., 100 mL vials), as is shown in FIG. 1B. A Tangential Flow Filtration (TFF) step was added to the process before the DNA was lyophilized using a freeze-drying program. After the mixing of DNA and PPC solutions (as described in Example 2), the 0.15 mg/mL formulated complex solution was processed through a KrosFlow KMPi system. This resulted in a DNA and PPC complex formulation that was up to five times more concentrated than the original 0.15 mg/mL formulation (0.75 mg/mL).

After the TFF step, the concentrated formulation was lyophilized using a freeze-drying program or stored frozen at <−20° C.

KMPi system configuration options for TFF are described in Table 1.

TABLE 1
Configuration of KMPi TFF system with a filter cut-off size of ≤50 kDa

| Parent | | | | |
|---|---|---|---|---|
| Window | Option | Description/Options | Settings | |
| KMPi Pump | Pump Mode | Controls the mode of operation of the | C Mode | |
| Control | | system | | |
| | Flowrate | Flowrate main pump | 1500-2500 | mL/min |
| | Conc 1 (CF) | Final desired concentration factor when | 5 | |
| | | operating in concentration mode | | |
| | Feed Weight | Initial weight of unconcentrated solution | 5-20 | kg |
| | | in feed bottle | | |
| | Valve 1 | Target pressure as controlled by pinch | 1.0-3.0 | psi |
| | | valve | | |
| | Direction | Direction of pump head | CW or CCW | |
| Main Pump | Tubing size | Size of tubing used with pump head | #73 | |
| | Ramp Rate | Time period over which pump is | 3 | s |

| | | accelerated to full speed | |
|---|---|---|---|
| | Slow for | | 10%/sec |
| | valve | | |
| | Control Shear | | On |
| Pressure and | Pressure Unit | Units of pressure readback | Psi |
| Valve | Valve Mode | Controls whether pinch valve is regulated | Auto |
| | | automatically by system or set to a manual | |
| | | fixed value by user | |
| | Tubing Size | Size of tubing installed on pinch valve | #73 |
| | Control | Controls which pressure sensor readback | TMP |
| | | is used to automatically control pinch- | |
| | | valve position | |
| | Start Position | Controls valve position at system startup | Custom or |
| | | | Open |
| | Custom Start | Allows for custom specification of initial | 20-70% |
| | Position | valve position (requires ‘Start Position’ | |
| | | setting of ‘Custom’) | |
| | Valve Size | Valve specification | KMPi |

### Example 4: Measurement of the Particle Size and Osmolality of Concentrated Liquid Formulations of Condensed Nucleic Acid with a Cationic Lipopolymer

Formulations of plasmid DNA with cationic lipopolymer (PPC) were prepared as described in Examples 1-3. For polymer/nucleic acid particle size measurement, an aliquot of the liquid formulation was analyzed using 90Plus/BI-MAS Particle Sizer from BROOKHAVEN INSTRUMENTS Corp., Holtsville, N.Y. Specifically, 50 μl of formulation is added to 950 μl of milli-Q water in polystyrene cuvets for analysis. For polymer/nucleic acid particle osmolality measurement, Pharmacopeia <785> method on osmolality and osmolarity was followed.

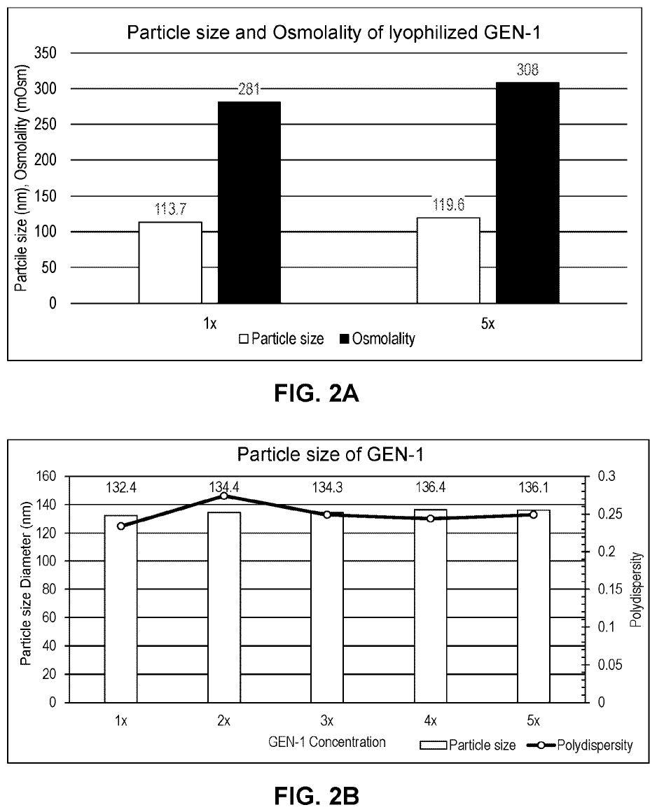

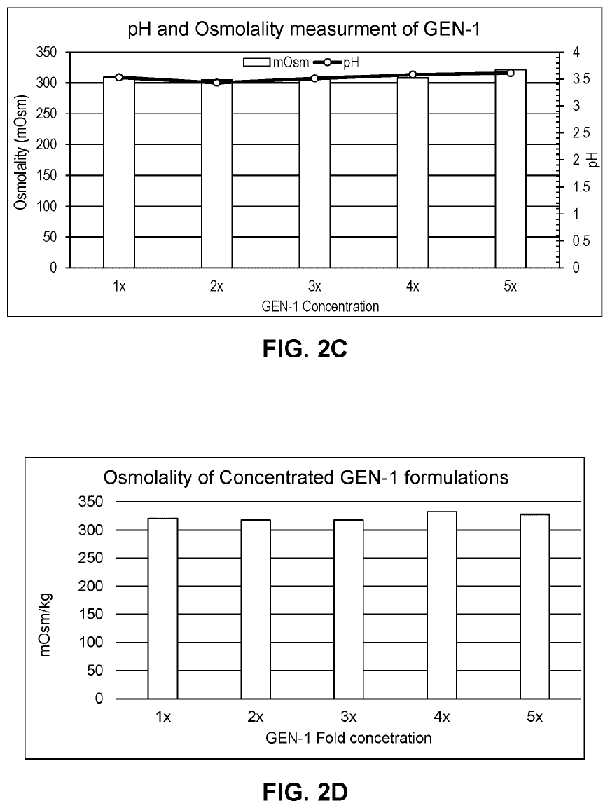

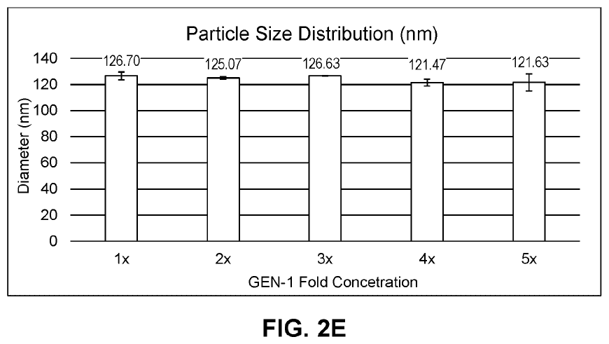

FIGS. 2A-2E illustrate the particle size and osmolality of DNA/PPC complexes from the lyophilized formulations at 1× (0.5 mg/mL DNA), 2× (1 mg/mL DNA), 3× (1.5 mg/mL DNA), 4× (2 mg/mL DNA), and 5× (2.5 mg/mL DNA) concentrations. Lyophilized formulation at higher concentrations did not significantly influence the particle size and osmolality, showing that the complexes were stable.

### Example 5: Measurement of Nucleic Acid Concentration and PPC in Concentrated Liquid Formulation of Nucleic Acid with a Cationic Lipopolymer

The amount of nucleic acid in highly concentrated formulations of DNA and PPC complexes were quantified using an AGILENT 8453 spectrophotometer (AGILENT TECHNOLOGIES, Inc. Santa Clara, CA). 50 μl of formulation is diluted with 950 μl water for injection (WFI) in a quartz cuvette and absorbance is measured using 260 nm wavelength. DNA concentration is determined assuming 1 Optical density (at 260 nm)=50 μg/mL of DNA. The amount of PPC in highly concentrated formulations of DNA and PPC complexes was measured using TNBSA (2,4,6-Trinitrobenzene Sulfonic Acid) assay reagent from Fisher Scientific (cat #TS-28997). TNBSA was used to measure free amino groups in PPC which form a highly chromogenic derivative and can be measured at 335 nm using UV/Vis Spectrophotometric.

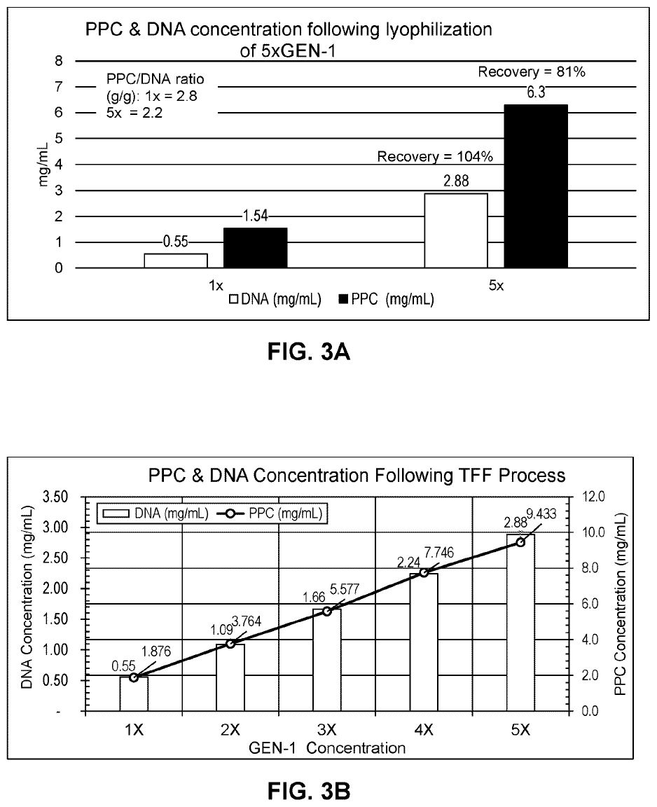

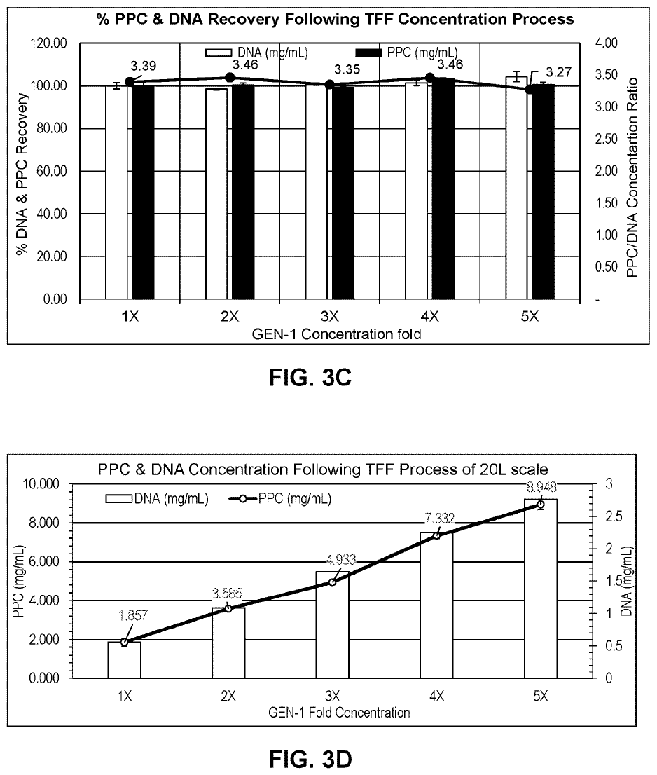

FIGS. 3A-3D illustrates DNA/PPC concentrations and ratio of lyophilized formulation without TFF process (1×) and with TFF process (2×-5× concentrated). Lyophilized formulation of higher concentrations produced using the TFF process did not significantly influence the DNA/PPC concentration ratio and percent recovery post-TFF process.

### Example 6: Analysis of the Nucleic Acid Condensation of Concentrated Liquid Formulations of Nucleic Acid with a Cationic Lipopolymer

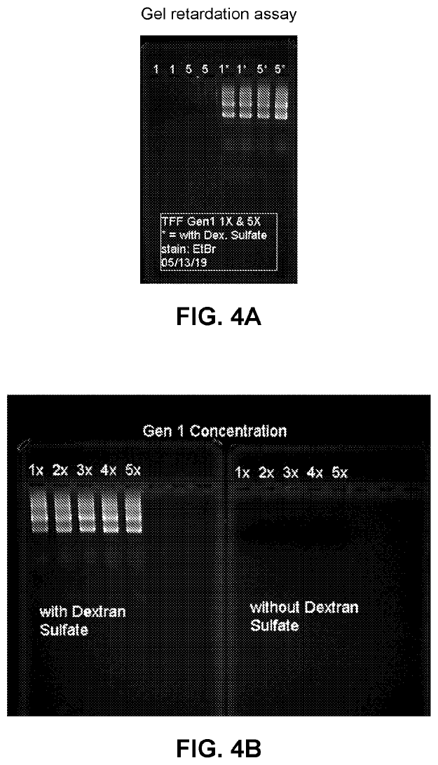

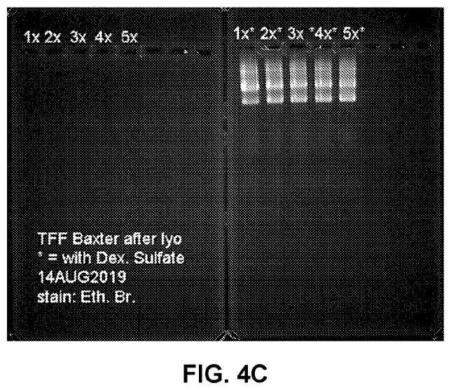

The ability of PPC polymer to condense plasmid DNA was evaluated in this example. Formulations of plasmid DNA with cationic lipopolymer, PPC, were prepared as described in Examples 1-3. The nucleic acid/polymer complexes (1×-5×) were electrophoresed using 1% agarose gel. The electrostatic attraction of negatively charged plasmid DNA to the positively charged PPC polymer prevented the DNA from traveling through the agarose gel. As shown in FIGS. 4A-4C, the DNA present in the highly concentrated formulations that underwent TFF process (2×-5×) were condensed and released in a similar manner as the 1× concentration when dextran sulfate was added.

### Example 7: Measurement of Transfection Activity of Concentrated Liquid Formulations of Nucleic Acid with a Cationic Lipopolymer

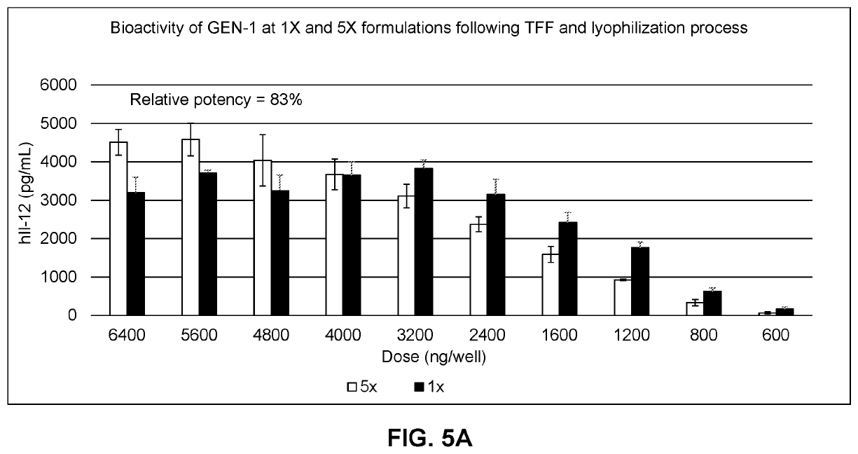

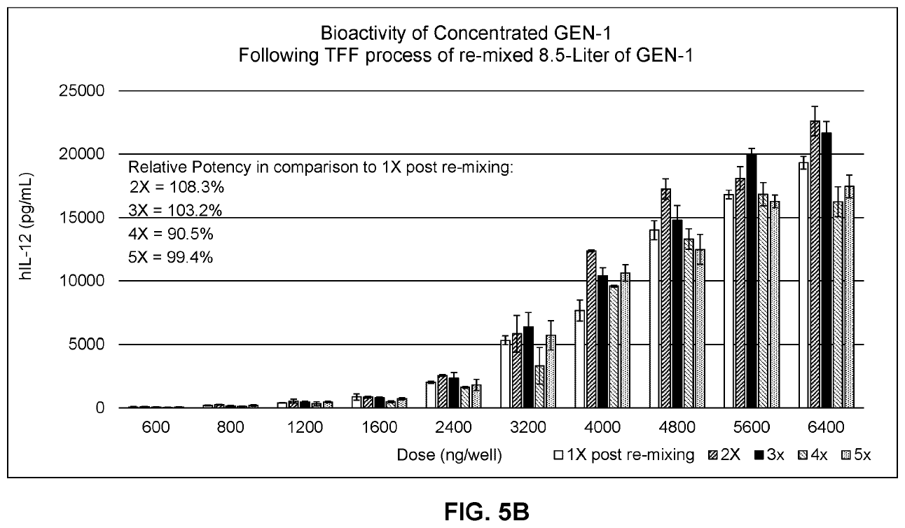

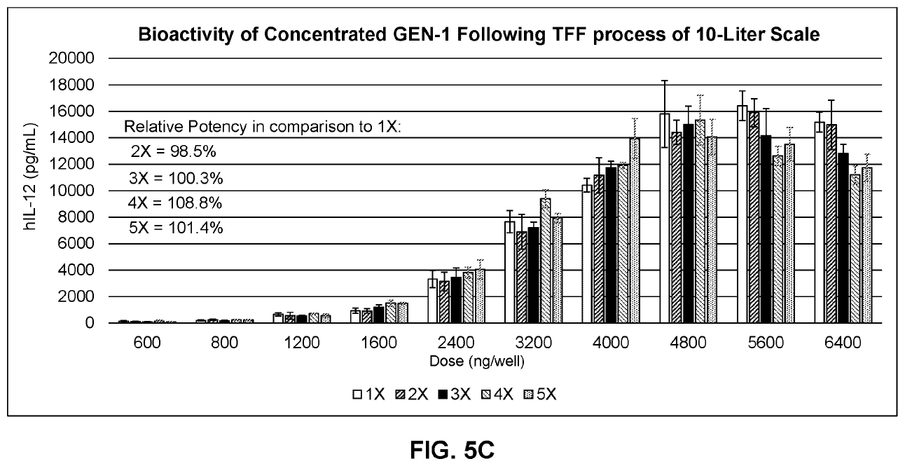

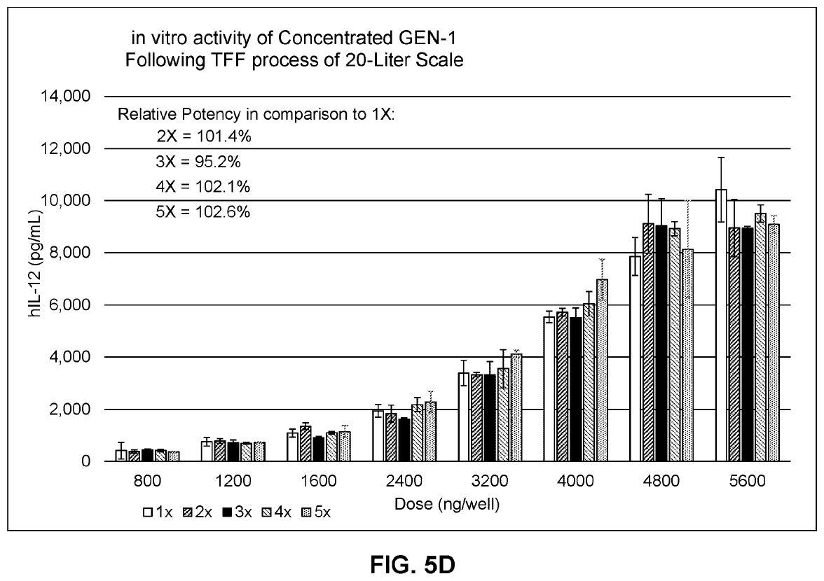

The transfection activity of formulations of DNA and PPC complexes was determined in vitro. Cos-1 cells (1.5×10⁵ cell/well) were seeded into 12-well tissue culture plates in 10% fetal bovine serum (FBS). Each well was incubated for 6 hours with 4 μg of complexed DNA in absence of FBS in a total volume of 500 μl of Dulbecco/Vogt Modified Eagle's Minimal Essential Medium (DMEM). When the incubation period was concluded, medium was replaced with 1 mL fresh DMEM supplemented with 10% FBS for another 40 hours. At the end of the incubation period, transfection activity was measured in the cell culture medium (IL-12). For measurement of IL-12 levels, cell culture medium was directly analyzed by an IL-12 ELISA assay. Direct comparison was made to that of 1× concentrated formulation. Transfection complexes containing IL-12 plasmid were prepared by methods described in Examples 1-3, and reconstituted at DNA concentrations ranging from 600 ng/well to 6400 ng/well from 1× to 5× concentrations. The levels of IL-12 expression from concentrated formulations of IL-12 plasmid/PPC complexes are shown in FIGS. 5A-5D. The data shows transfection biological activity of nucleic acid complexes in highly concentrated form was preserved.

### Example 8: IL-12 Expression in Normal Brain Parenchyma after Intracranial Expression of Concentrated Liquid Formulations of Nucleic Acid with Cationic Lipopolymer

Direct administration of IL-12 plasmid with cationic polymer, PPC, in normal brain tissue was examined to determine if highly concentrated formulation of nucleic acid and cationic lipopolymer was biologically active in vivo. Immunohistochemical staining for IL-12 was performed on slices of brains from animals euthanized 14 days or 1 month after treatment. Brain parenchyma of animals treated with PPC alone did not show any IL-12 staining. In contrast, brain parenchyma of mice injected with pmIL-12/PPC intracranially stained positive for IL-12. This experiment demonstrates biological activity of nucleic acid complexes with a cationic polymer is preserved during the concentration process. In addition, it was shown that the IL-12 remains present for at least a month after injection. Moreover, the presence of this cytokine in the brains of animals that remained alive until euthanized suggests that the actual expression of IL-12 did not cause lethal toxicity in brain.

### Example 9: Biological Activity of Concentrated Liquid Formulations of Nucleic Acid with Cationic Lipopolymer in Tumor Bearing Mice

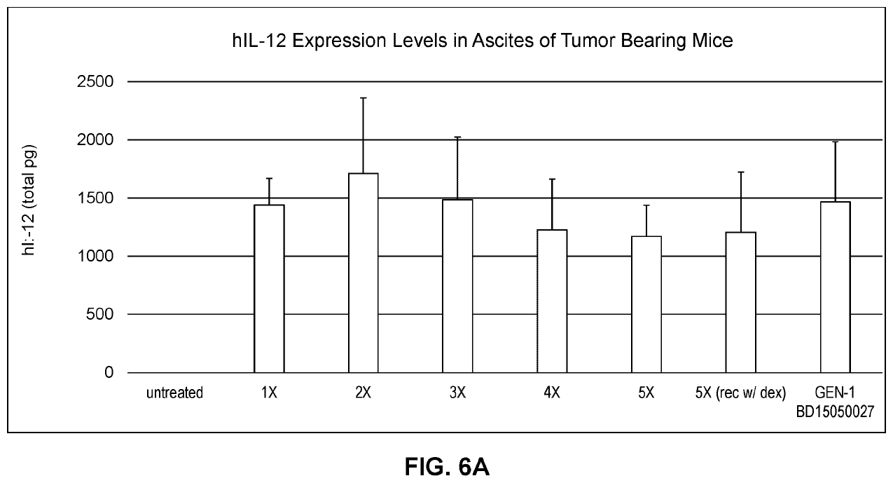

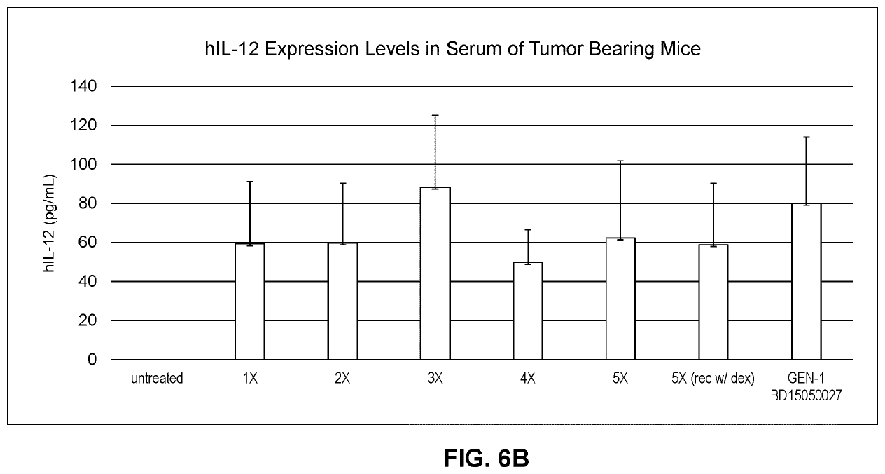

The biological activity of formulations (1×-5×) of fully condensed nucleic acid expressing IL-12 gene were examined in ascites and serum of tumor bearing mice, as shown in FIG. 6A and FIG. 6B. Tumors were implanted by intraperitoneal injection of 2.5×10⁶ ID8 ovarian cancer cells into C57BL/6 female mice. At 41 days post-tumor implant, once the animals reached a significant increase in body weight vs their starting weight, mice were randomized into treatment groups. At 43 days post tumor implant, GEN-1 was administered by intraperitoneal injection at the dose of 10 mg/kg. Approximately 24 h after injection of GEN-1 administration, animals were euthanized for serum and ascites collection. Animals were weighed before and after complete removal of ascites and the volume of ascites was calculated. The hIL-12 expression levels were measured in both serum and ascites using human IL-12 p70 ELISA kit. These data demonstrated that the highly concentrated formulations of IL-12 nucleic acid, when compared to 1× formulation, maintained gene expression for in vivo application.

### Example 10: Biological Activity of Concentrated Liquid Formulations of Nucleic Acid with Cationic Lipopolymer in Tumor Bearing Mice

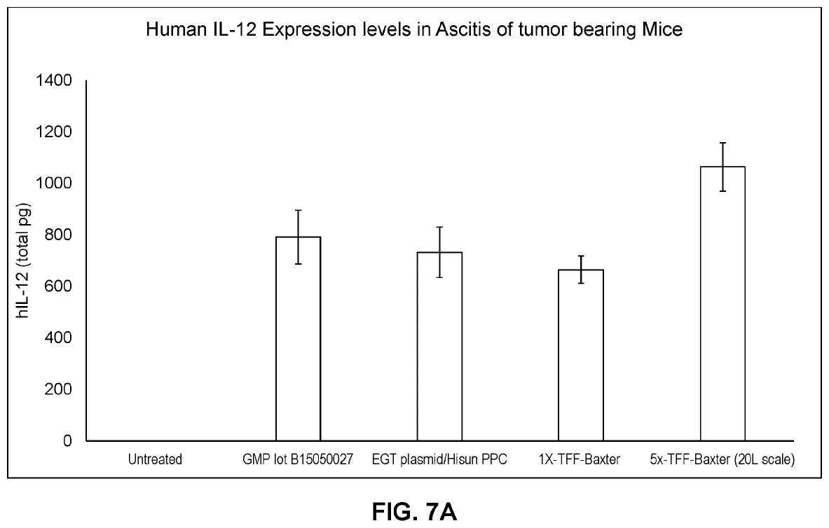

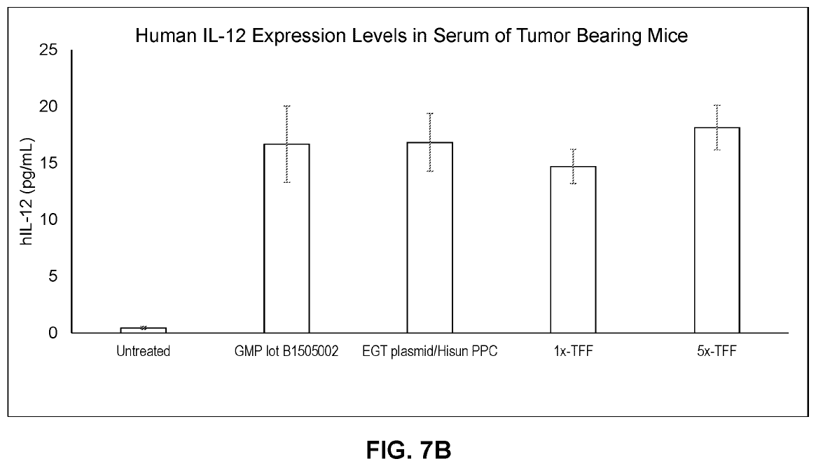

The biological activity of fully condensed nucleic acid expressing IL-12 gene at 1× and 5× formulations post-TFF and formulations from two separate lots with no TFF were examined in ascites and serum of tumor bearing mice, as shown in FIG. 7A and FIG. 7B. Tumors were implanted by intraperitoneal injection of 2.5×10⁶ ID8 ovarian cancer cells into C57BL/6 female mice. At 41 days post-tumor implant, once the animals reached a significant increase in body weight vs their starting weight, mice were randomized into treatment groups. At 43 days post tumor implant, GEN-1 was administered by intraperitoneal injection at the dose of 10 mg/kg. Approximately 24 h after injection of GEN-1 administration, animals were euthanized for serum and ascites collection. Animals were weighed before and after complete removal of ascites and the volume of ascites was calculated. The hIL-12 expression levels were measured in both serum and ascites using human IL-12 p70 ELISA kit. These data demonstrated that the highly concentrated formulation of IL-12 nucleic acid, when compared to 1× formulation, maintained gene expression for in vivo application.

### Example 11: Long-Term Stability of the Lyophilized or Concentrated Liquid Formulations of Nucleic Acid with Cationic Polymer

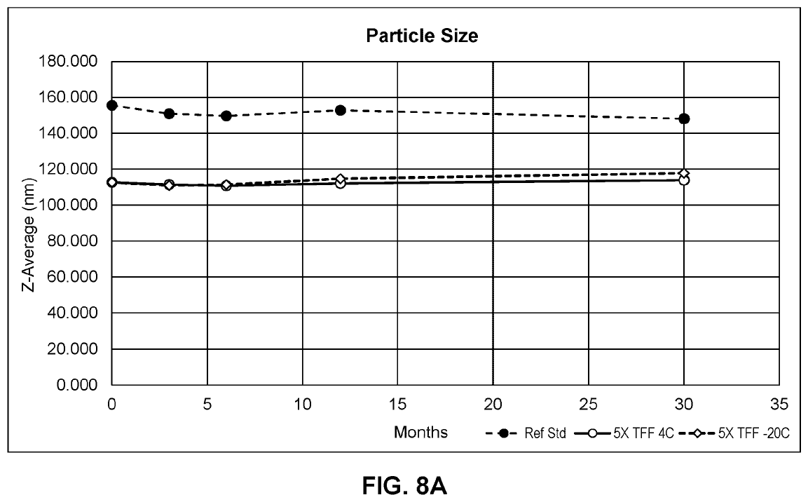

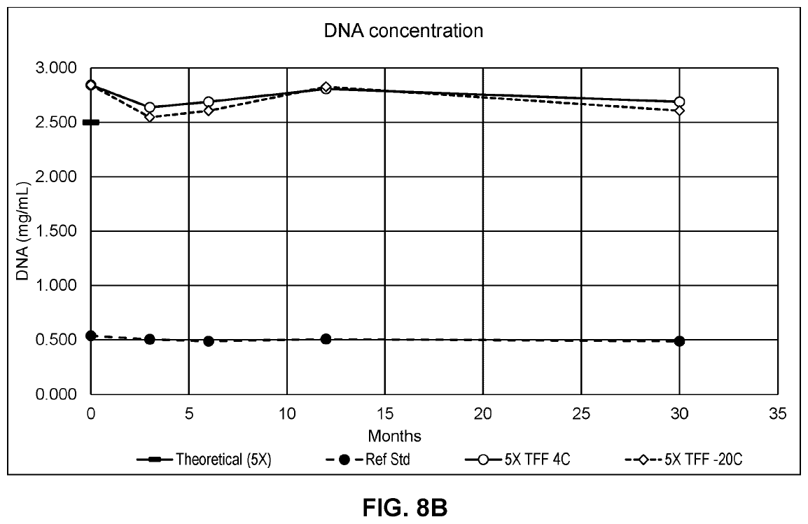

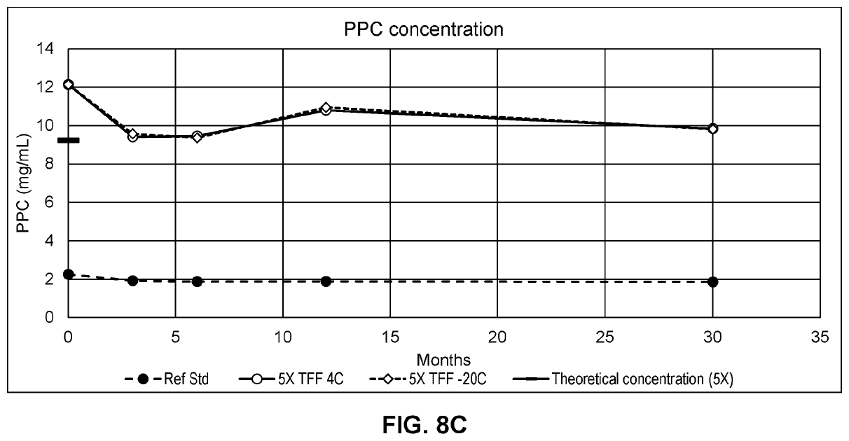

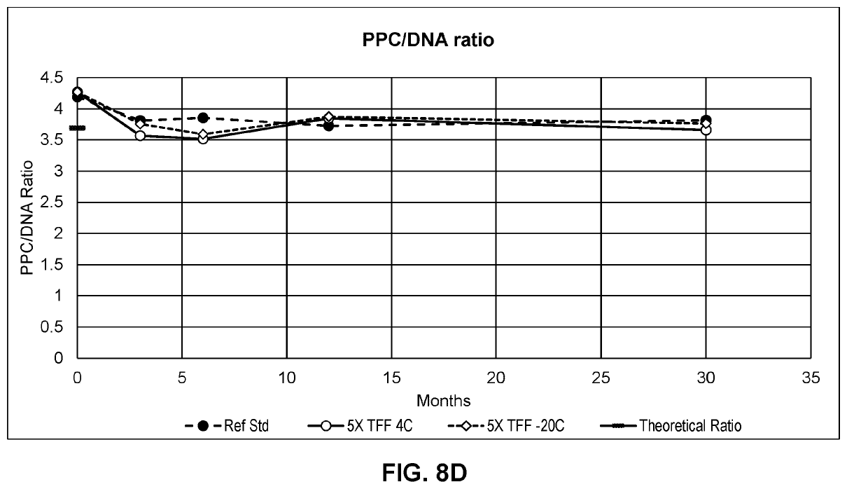

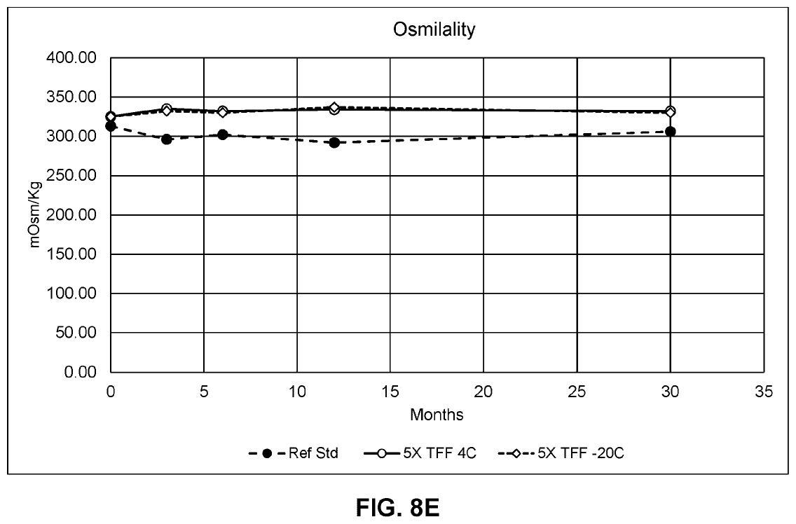

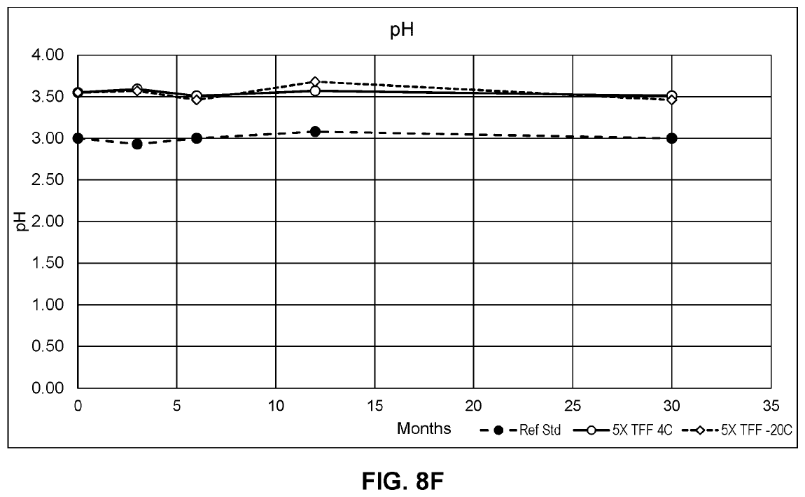

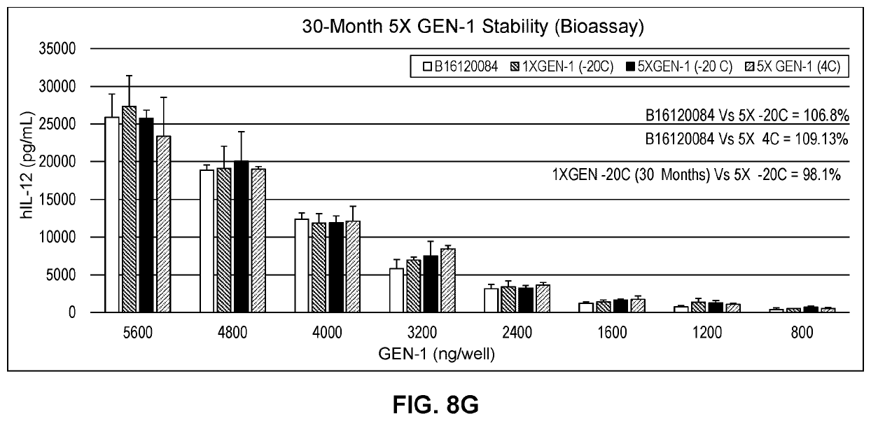

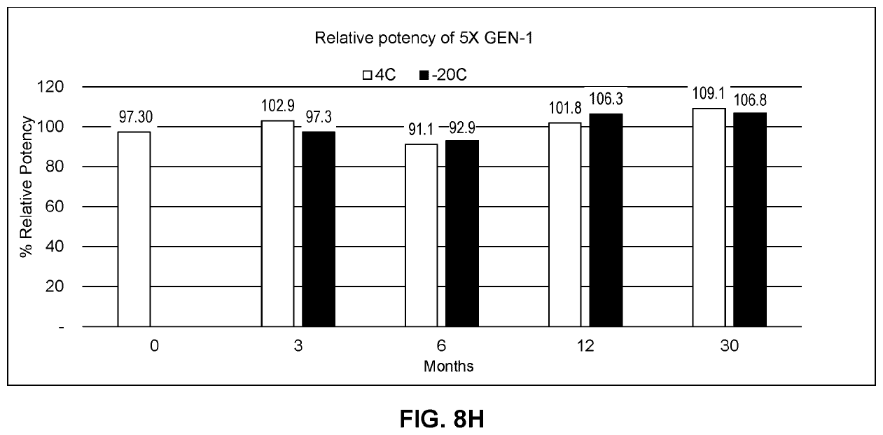

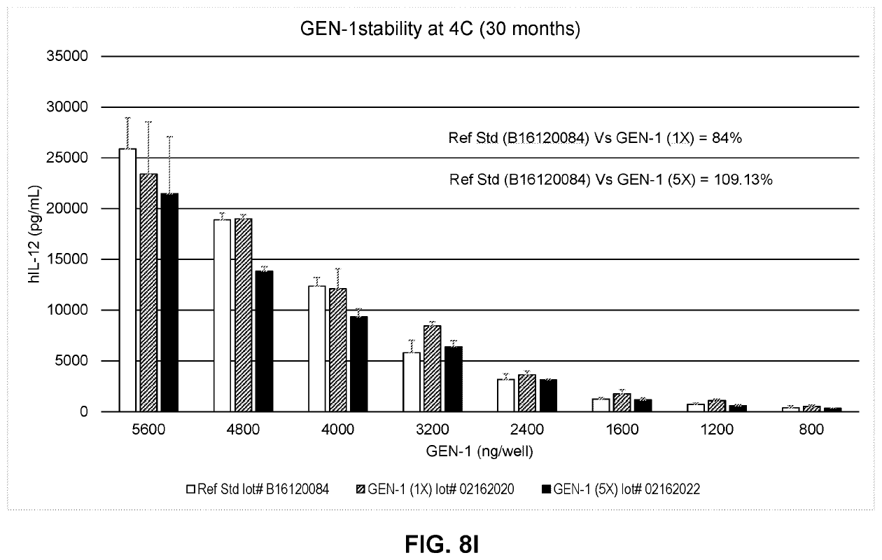

Lyophilized IL-12/PPC complexes were prepared using the method outlined in Examples 1-3 and stored at −80° C., −20° C., 4° C., and 25° C. At the time of analysis, vials were removed from storage and 2.4 mL of water for injection (WFI) was added. For each sample, pH, DNA concentration, osmolality, particle size and biological activity were measured. As shown in FIG. 8A-8F, the DNA particle size, DNA concentration, PPC concentration, PPC/DNA ratio, osmolality, and pH of the 5× concentrated IL-12/PPC complex was maintained after storage of up to 30 months at the indicated temperatures, when compared to reference standard of 1× concentrated IL-12/PPC complex. The gene transfer activity of pIL-12/PPC was quantified in COS-1 cells as described in Example 7. The COS-1 cells were transfected with the biological material at 4 μg DNA. The levels of IL-12 in cell culture media were quantified 48 hours after the transfection with a commercially available ELISA kit. The bioactivity results from the 30 month stability study are illustrated in FIG. 8G. There was no significant change in bioactivity of the biological product of the 5× concentrated IL-12/PPC complex during the storage period at −20° C. or 4° C.

The relative potency results of up to 30 months are illustrated in FIG. 8H and FIG. 8I. The relative potency of 5× concentrated IL-12/PPC complex, during the storage period at −20° C. or 4° C., also showed no significant decline in DNA particle size, DNA concentration, PPC concentration, PPC/DNA ratio, osmolality, and pH after storage of up to 30 months when compared with the 1× concentrated IL-12/PPC complex stored at −80° C. Thus, these result show that the 5× concentration was able to maintain stability of particle size, DNA concentration, PPC concentration, PPC/DNA ratio, osmolality, pH, and biological activity at 4° C. and −20° C. during 30-month storage period. Furthermore, the results show superior relative potency of 5× formulation compared to the 1× formulation after 30-month storage at 4° C.

It is to be understood that the above-described compositions and modes of application are only illustrative of embodiments of the invention. Numerous modifications and alternative arrangements may be devised by those skilled in the art without departing from the spirit and scope of the invention and the appended claims are intended to cover such modifications and arrangements. Thus, while certain aspects of the invention have been described above, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A method of making a concentrated nucleic acid composition comprising:
(a) combining (i) a DNA plasmid comprising a nucleic acid encoding a human IL-12 polypeptide, (ii) a cationic lipopolymer comprising polyethylene glycol (PEG)-polyethylenimine (PEI)-cholesterol (PPC), and (iii) a filler excipient in an aqueous medium, wherein the nucleic acid complexes with the cationic lipopolymer, thereby forming a nucleic acid mixture having a concentration of at least 0.1 to 0.2 mg/ml; and
(b) concentrating the nucleic acid mixture of (a) by tangential flow filtration to form a concentrated nucleic acid composition having a concentration of at least 0.7 to 0.8 mg/mL, wherein the percent recovery of nucleic acid complexed with cationic lipopolymer after tangential flow filtration is at least 75%, at least 80%, at least 85%, at least 90%, at least 95%, or 100%, and wherein the concentrated nucleic acid composition is suitable for pharmaceutical use, storage at about −20° C. or less, storage at about 4° C. or less, and/or lyophilization.

2. The method of claim 1, further comprising storing the concentrated nucleic acid composition at about −20° C. or less or about 4° C. or less for at least 24 hours, at least one week, at least one month, at least one year, or at least 30 months.

3. The method of claim 1 or 2, wherein the nucleic acid mixture has a concentration of at least 0.15 mg/mL.

4. The method of any one of claims 1-3, wherein the concentrated nucleic acid composition has a concentration of at least 0.75 mg/mL.

5. The method of any one of claims 1-4, further comprising (c) lyophilizing the concentrated nucleic acid composition having a concentration of at least 0.75 mg/mL in a volume of 20-40 mL, thereby forming a lyophilized formulation comprising at least 10 mg, at least 15 mg, at least 20 mg, at least 25 mg, or at least 30 mg of the DNA plasmid.

6. The method of claim 5, wherein the lyophilized formulation comprises about 10-50 mg of the DNA plasmid.

7. The method of claim 5 or 6, further comprising (d) reconstituting the lyophilized formulation in a diluent thereby forming a reconstituted composition.

8. The method of claim 7, wherein the reconstituted composition comprises about 0.1 mg/mL to about 20 mg/mL of the DNA plasmid.

9. The method of claim 7, wherein the diluent is water, about 5% dextrose, or saline.

10. The method of any one of claims 1-9, wherein the DNA plasmid further comprises a promoter, preferably a cytomegalovirus (CMV) promotor.

11. The method of any one of claims 1-10, wherein the nucleic acid mixture comprises GEN-1 nanoparticles.

12. The method of any one of claims 1-11, wherein the ratio of amine nitrogen in the cationic polymer backbone to phosphate in the nucleic acid is from about 10:1 to about 100:1.

13. The method of any one of claims 1-12, wherein the filler excipient comprises a sugar, a sugar alcohol, a starch, a cellulose, or combinations thereof.

14. The method of any one of claims 1-13, wherein the filler excipient comprises one or more of lactose, sucrose, trehalose, dextrose, galactose, mannitol, maltitol, maltose, sorbitol, xylitol, mannose, glucose, fructose, polyvinyl pyrrolidone, glycine, maltodextrin, hydroxymethyl starch, gelatin, sorbitol, ficol, sodium chloride, calcium phosphate, calcium carbonate, and/or polyethylene glycol.

15. The method of any one of claims 1-14, wherein the filler excipient comprises lactose.

16. A pharmaceutical composition comprising the concentrated nucleic acid composition of any one of claims 1-15 or the reconstituted composition of any one of claims 7-15.

17. The pharmaceutical composition of claim 16 which comprises greater than 6 mg of DNA plasmid in a volume of less than 50 mL.

18. The pharmaceutical composition of claim 16 or 17 which comprises 10-40 mg of DNA plasmid in a volume of less than 50 mL.

19. The pharmaceutical composition of any one of claims 16-18 which comprises about 30 mg of DNA plasmid in a volume of 20-45 mL.

20. The pharmaceutical composition of any one of claims 16-19 which is used to prepare a dosage of 100-200 mg of plasmid DNA in less than 300 mL of aqueous solution.

21. The pharmaceutical composition of any one of claims 16-20, wherein the DNA particle size, concentration, osmolality, and pH is maintained after storage at about −20° C. or about 4° C. for up to at least 30 months.

22. A method of treating cancer in a subject comprising administering the pharmaceutical composition of any one of claims 16-21 to a subject in need thereof.

23. The method of claim 22, wherein the pharmaceutical composition is formulated for intratumoral, intraperitoneal, intravesicle, intravenous, intra-arterial, intratracheal, intrahepaticportal, intracranial, intramuscular, or intraarticular administration.

24. The method of claim 22 or 23, wherein the cancer is ovarian cancer.

25. The method of any one of claims 22-24, further comprising administering a chemotherapeutic agent to the subject.

26. A kit comprising a first vial comprising a nucleic acid mixture comprising (i) a DNA plasmid comprising a nucleic acid encoding a human IL-12 polypeptide, (ii) a cationic lipopolymer comprising polyethylene glycol (PEG)-polyethylenimine (PEI)-cholesterol (PPC), and (iii) a filler excipient in an aqueous medium, wherein the nucleic acid is complexed with the cationic lipopolymer and the nucleic acid mixture comprises at least 25 mg to 50 mg of the DNA plasmid, and optionally further comprising a second vial comprising a diluent.

27. The kit of claim 26, wherein the first vial and/or second vial can hold up to about 100 mL, about 150 mL, about 200 mL, about 250 mL or about 500 mL.

28. The kit of claim 26 or 27, wherein the second vial comprises a volume of about 200-300 mL of diluent.

29. The kit of any one of claims 26-28, wherein the first vial comprises about 10-50 mg, about 10-40 mg, about 20-50 mg, about 20-25 mg, about 20-45 mg, about 20-40 mg, or about 25-35 mg of the DNA plasmid.

30. A kit comprising a vial comprising a nucleic acid mixture comprising (i) a DNA plasmid comprising a nucleic acid encoding a human IL-12 polypeptide, (ii) a cationic lipopolymer comprising polyethylene glycol (PEG)-polyethylenimine (PEI)-cholesterol (PPC), and (iii) a filler excipient in an aqueous medium, wherein the nucleic acid is complexed with the cationic lipopolymer; and wherein the nucleic acid mixture is in an aqueous solution at a concentration of at least 0.7 mg/mL to 0.8 mg/L (e.g., at least 0.75 mg/mL).

31. The kit of claim 30, wherein the nucleic acid mixture is in an aqueous solution at a concentration of at least 0.75 mg/mL.

32. The kit of any one of claims 30-31, wherein the vial first vial and/or second vial can hold up to about 100 mL, about 150 mL, about 200 mL, about 250 mL or about 500 mL.

33. The kit of any one of claims 30-32, wherein the vial further comprises a volume of about 200-300 mL of diluent.

34. The kit of any one of claims 30-33, wherein vial comprises 10-50 mg, 10-40 mg, 20-50 mg, 20-45 mg, 20-40 mg, 20-25 mg, or 25-35 mg of the DNA plasmid.

35. The kit of any one of claims 26-34, wherein the kit can be stored at −20° C. or less.

36. The kit of any one of claims 26-35, wherein the composition is prepared according to the methods of any one of claims 1-15 or the pharmaceutical composition of any one of claims 16-21.
